(19) United States
(12) Patent Application Publication
(10) Pub. No.: US 2023/0331869 A1
(43) Pub. Date: Oct. 19, 2023

(54) FIBRIN AND B-CELL LYMPHOMA

(71) Applicant: The J. David Gladstone Institutes, a testamentary trust established under the will of J. David, San Francisco, CA (US)

(72) Inventors: Katerina Akassoglou, San Francisco, CA (US); Jae Kyu Ryu, San Francisco, CA (US)

(21) Appl. No.: 18/116,226

(22) Filed: Mar. 1, 2023

Related U.S. Application Data
(60) Provisional application No. 63/315,527, filed on Mar. 1, 2022.

(51) Int. Cl. C07K 16/36; A61P 35/00

(52) U.S. Cl. CPC C07K 16/36; A61P 35/00; C07K 2317/565; C07K 2317/24

(57) ABSTRACT

Provided herein is a novel approach to treating CNS tumors. Specifically, the data shows that fibrin therapies targeting fibrin, for example the γ³⁷⁷⁻³⁹⁵ fibrin epitope, are novel CNS tumor therapeutics e.g., against primary CNS lymphoma

### PRIORITY APPLICATION

This application claims the benefit of priority to U.S. Provisional Patent Application Ser. No. 63/315,527, filed Mar. 1, 2022, the content of which is incorporated herein by reference in its entirety.

### GOVERNMENT SUPPORT

This invention was made with government support under grant nos. R01NS052189 awarded by The National Institutes of Health. The government has certain rights in the invention.

### INCORPORATION BY REFERENCE OF SEQUENCE LISTING

A Sequence Listing is provided herewith as an xml file, “2349661.xml” created on Jul. 7, 2023 and having a size of 59,856 bytes. The content of the xml file is incorporated by reference herein in its entirety.

### BACKGROUND

Central nervous system (CNS) lymphoma is an extranodal non-Hodgkin B-cell lymphoma characterized by malignant lymph tissue arising in the brain or spinal cord, associated with inflammation and blood brain barrier (BBB) disruption.

### SUMMARY

Although BBB disruption is known to occur in patients with CNS lymphoma, a direct link between these two has not been shown. Herein, abundant deposition of the blood coagulation protein fibrinogen around B-cell lymphoma was detected in CNS lymphoma patients and in the CNS parenchyma in an orthotopic mouse model. Functional enrichment analysis of unbiased cerebrospinal fluid proteomics of CNS B-cell lymphoma patients showed that coagulation protein networks were highly connected with tumor-associated biological signaling pathways. In vivo two-photon imaging demonstrated that lymphoma growth was associated with BBB disruption, and in vitro experiments identified a role for fibrinogen in promoting lymphoma cell adhesion. Overall, these results identify perivascular lymphoma clustering at sites of fibrinogen deposition, and demonstrate that fibrinogen is a target for pharmacologic intervention in metastatic B-cell lymphoma associated with BBB disruption.

One embodiment provides a method to treat or prevent cancer comprising administering anti-fibrin/anti-fibrinogen antibodies to a subject in need thereof. In one embodiment, the cancer is a cancer of the central nervous system (CNS). In another embodiment, the CNS cancer has blood brain barrier (BBB) disruption. In one embodiment, there is malignant tissue in brain and/or spinal cord of the subject. In one embodiment, the CNS cancer is a lymphoma, such as B-cell lymphoma. In one embodiment, the CNS cancer is metastatic.

In one embodiment, the antibodies are human antibodies or humanized antibodies. In one embodiment, the anti-fibrin antibodies/anti-fibrinogen antibodies comprise a CDR region with a sequence comprising SEQ ID NOs:10-12 and 14-16, or combination of CDR regions with sequences comprising SEQ ID NO:10, 11, 12, 14, 15, or 16. In another embodiment, the anti-fibrin antibodies/anti-fibrinogen antibodies comprise a SEQ ID NOs: 26 and 28.

One embodiment provides a method to block tumor cell interactions with fibrin comprising contacting one or more tumor cells with an anti-fibrin/anti-fibrinogen antibodies. In one embodiment, the antibodies are human antibodies or humanized antibodies. In one embodiment, the anti-fibrin antibodies/anti-fibrinogen antibodies comprise a CDR region with a sequence comprising SEQ ID NOs:10-12 and 14-16, or combination of CDR regions with sequences comprising SEQ ID NO:10, 11, 12, 14, 15, or 16. In another embodiment, the anti-fibrin antibodies/anti-fibrinogen antibodies comprise a SEQ ID NOs: 26 and 28.

One embodiment provides a of disrupting adhesion of lymphoma cells at a BBB disruption comprising contacting said cells with one or more an anti-fibrin/anti-fibrinogen antibodies. In one embodiment, the antibodies are human antibodies or humanized antibodies. In one embodiment, the anti-fibrin antibodies/anti-fibrinogen antibodies comprise a CDR region with a sequence comprising SEQ ID NOs:10-12 and 14-16, or combination of CDR regions with sequences comprising SEQ ID NO:10, 11, 12, 14, 15, or 16. In another embodiment, the anti-fibrin antibodies/anti-fibrinogen antibodies comprise a SEQ ID NOs: 26 and 28.

### BRIEF DESCRIPTION OF THE DRAWINGS

FIGS. 1A-1B. Coagulation factors are required for lymphoma formation in the brain. A: Network of co-expressed proteins in cerebrospinal fluid from cancer patients. Nodes (circles) of the network represent proteins, and the edges (purple lines) between two nodes indicate published evidence for co-expression. Nodes are colored on the basis of functional categories significantly enriched in Reactome pathway terms (false discovery rate (FDR), <0.05). B: Tissue array of 100 diffuse large B-cell lymphomas in specified tissues. Immunohistochemistry of fibrinogen deposits in specified tissue array comprising 100 malignant diffuse large B-cell lymphomas. Representative fibrinogen-staining tissues from lymph node and brain are shown. Data are given as means±SEM (B). GPCR, G protein-coupled receptors; IR, immunoreactivity.

FIGS. 2A-2C. Fibrinogen is associated with central nervous system (CNS) lymphoma. Microscopy of tissue sections from postmortem brains of human patients with CNS lymphoma, stained with antibodies to fibrinogen, laminin (basal membrane), CD20 (B cell), and ionized calcium-binding adaptor protein-1 (Iba-1; microglia), showing fibrinogen surrounding a CD20⁺ B-cell lymphoma around leaky blood vessels. Scale bars: 100 mm (A); 80 mm (B and C).

FIGS. 3A-3E. Central nervous system (CNS) lymphoma is associated with leaky blood-brain barrier (BBB). A-D: Microscopy of tissue sections of mouse brain implanted with B-cell lymphoma derived from CNS lymphoma patients, stained with antibodies to fibrinogen, CD31 (endothelial cells), and CD20 (B cell), showing patient-derived xenografted lymphoma cells are accumulated around leaky BBB. E: Top panels: In vivo two-photon (2P) brain imaging of BBB leaks (detection of Alexa 647elabeled dextran) in the cortex of a mouse brain implanted with B-cell lymphoma Raji cells. Bottom panel: Quantification of extravascular dextran in cortex. Data are given as means±SEM (E, bottom panel). n=3 mice (E, bottom panel). Scale bars: 150 mm (A-C); 50 mm (D and E).

FIGS. 4A-4D. In vivo two-photon (2P) imaging of parenchymal movement of brain implanted Raji cells in regions with and without blood-brain barrier (BBB) leakage. A: In vivo 2P time-lapse images, showing tracking of intracortical movement of Raji cells in Raji cell-implanted mice over 45 minutes. White tracks indicate cells showing movement over the 45-minute imaging interval. B: Quantification of traveled distance measured over 45 minutes, shown in A. Data are shown for individual cells (five cells per mouse) and as averages of five cells per mouse. C: Lymphoma cell adhesion assay. Quantification of adhered lymphoma cells labeled with Calcein-AM on different coating conditions: uncoated, fibrinogen, and albumin. D: Quantification of adhered lymphoma cells to coated plasma isolated from wild-type and Fga^{-/-} mice. Data are given as means±SEM (C and D). n=3 mice with BBB leakage and without BBB leakage (B); n=3 independent experiments (C and D). *P<0.05, **P<0.01, ***P<0.001, and ****P<0.0001 (two-way analysis of variance with the Tukey multiple comparisons test). Scale bar=25 mm (A). Ex/em, excitation/emission.

FIG. 5. Lymphoma cell adhesion assay. Quantification of adhered lymphoma cells labeled with Calcein-AM to coated plasma isolated from wild-type and Fgg^{390-396A} mice. N=4 independent experiments. **P<0.01 and ****P<0.0001 (one-way analysis of variance with Tukey multiple-comparison test). Ex/em, excitation/emission. This data shows quantification of adhered human lymphoma cells to coated plasma isolated from wild type and Fgg^{390-396A} mice. This data shows that fibrinogen signaling through CD11b/CD18 regulates adhesion of lymphoma cells at sites of BBB disruption.

FIG. 6. Over time, intravascular fluorescent dextran gradually leaked into the perivascular tissue that was occupied by lymphoma cells.

FIGS. 7A-7D. Fibrinogen depletion using ancrod reduces pro-inflammatory and oncogenic responses in human B-cell lymphoma in vivo. A. Experimental design diagram: Rag2^{-/-}γc^{-/-} mice were implanted with ancrod-filled nitro-osmotic pumps or with saline-filled pumps. Two days after pump implantation, human B-cell lymphoma cells were stereotaxically injected into the brain. Ten days after tumor cell injection, tumor bearing brain tissues were prepared for further analysis. B. Fibrinogen depletion with ancrod specifically inhibits lymphoma-induced human IL-6 and Mmp3 gene upregulations. Data presented as mean±s.e.m. n=2 independent experiments. C. Fibrinogen depletion with ancrod blocks lymphoma derived PCNA, Myc, p-Foxo3a, p-GSK3b, and p-AKT expression in vivo. n=2 independent experiments. D. Fibrin activates oncogenic signaling in human B-cell lymphoma cells stimulated with fibrin. Data are representative of two independent experiments.

### DETAILED DESCRIPTION OF THE INVENTION

Provided herein is a novel approach to treating CNS tumors. Specifically, the data shows that fibrin therapies targeting fibrin, for example the γ³⁷⁷⁻³⁹⁵ fibrin epitope, are novel CNS tumor therapeutics e.g., against primary CNS lymphoma (PCNSL), a rare form of extranodal lymphoma that occurs in the brain, leptomeninges, and spinal cord. PCNSL is characterized with heterogeneous clinical symptoms, genomic variations, and histopathological subtypes. Patients undergoing treatment often suffer from long-term toxic effects that cause secondary malignancies and adversely affect their quality of life. There is an unmet medical need for less toxic, safer and more effective treatments for several CNS tumors. Strategies based on blocking tumor cell interactions (such as by a therapeutic targeting γ³⁷⁷⁻³⁹⁵ fibrin epitope (e.g., peptides, antibodies or small molecules) or by depleting fibrin in the CNS to target oncogenic functions of fibrin brain and other tumors) with fibrin offer new therapeutic interventions.

Provided herein is the description of how depletion of the γ³⁷⁷⁻³⁹⁵ fibrin epitope protects from the growth of B-cell lymphoma in the brain. Anti-fibrin tumor therapy would have the following advantages compared to other immunotherapy for tumor treatment: a) the approach utilizes a novel mechanism of action; no fibrin-targeting therapies are currently available for tumor treatment; b) blocking fibrin's interaction with a specific receptor on tumor cells is a highly selective intervention, and thus less likely to produce toxicity and/or side effects, c) understanding the molecular mechanisms involved provides new therapeutic avenues for treating other CNS brain tumors or injury, or diseases involving blood-brain barrier (BBB) disruption.

### Cancer

Provided herein are compositions and methods to prevent and/or treat central nervous system (CNS) cancers, including CNS lymphoma (e.g., PCNSL), which is a form of non-Hodgkin B-cell neoplasm that can occur as either a primary or a metastatic disease, or other brain cancers (such as glioblastoma). CNS lymphoma is an extranodal non-Hodgkin B-cell lymphoma characterized by malignant lymph tissue arising in the brain or spinal cord, associated with inflammation and blood brain barrier (BBB) disruption (e.g., leaky BBB).

### Fibrinogen/Cryptic Peptide

Fibrinogen is a glycoprotein complex, produced in the liver, that circulates in the blood of vertebrates. On activation of the coagulation cascade, such as during tissue and vascular injury, fibrinogen is converted enzymatically by thrombin to fibrin and then to a fibrin-based blood clot. Conversion of fibrinogen to fibrin is associated with exposure of a fibrin cryptic epitope. Fibrin can also bind and reduce the activity of thrombin (fibrin is sometimes referred to as antithrombin I), which limits clotting. Fibrin also mediates blood platelet and endothelial cell spreading, tissue fibroblast proliferation, capillary tube formation, and angiogenesis. Fibrin therefore can promote revascularization and wound healing. As demonstrated herein, fibrinogen/fibrin (e.g., deposition) plays a role in B-cell lymphoma/CNS lymphoma.

An example of a human fibrinogen sequence is the fibrinogen gamma chain isoform gamma-A precursor sequence (NCBI accession number NP_000500.2), provided as SEQ ID NO: 39 below.

| | |
|---|---|
| 1 | MSWSLHPRNL ILYFYALLFL SSTCVAYVAT RDNCCILDER |
| 41 | FGSYCPTTCG IADFLSTYQT KVDKDLQSLE DILHQVENKT |
| 81 | SEVKQLIKAI QLTYNPDESS KPNMIDAATL KSRKMLEEIM |
| 121 | KYEASILTHD SSIRYLQEIY NSNNQKIVNL KEKVAQLEAQ |
| 161 | CQEPCKDTVQ IHDITGKDCQ DIANKGAKQS GLYFIKPLKA |
| 201 | NQQFLVYCEI DGSGNGWTVF QKRLDGSVDF KKNWIQYKEG |
| 241 | FGHLSPTGTT EFWLGNEKIH LISTQSAIPY ALRVELEDWN |
| 281 | GRISTADYAM FKVGPEADKY RLTYAYFAGG DAGDAFDGFD |
| 321 | FGDDPSDKFF TSHNGMQFST WDNDNDKFEG NCAEQDGSGW |
| 361 | WMNKCHAGHL NGVYYQGGTY SKASTPNGYD NGIIWATWKT |
| 401 | RWYSMKKTTM KIIPFNRLTI GEGQQHHLGG AKQAGDV |

Antibodies directed against the fibrin γ epitope, KKTTMKIIPFNRLTIG (SEQ ID NO:2, highlighted above in the SEQ ID NO:1 sequence), can be effective for treating B-cell lymphoma.

Additional epitopes that can be targeted by anti-fibrinogen/fibrin antibodies include any of the Bβ119-129 (YLLKDLWQKRQ, SEQ ID NO: 3), γ163-181 (QSGLYFIKPLKANQQFLVY; SEQ ID NO: 4), γ364-395 (DNGIIWATWKTRWYSMKKTTMKIIPFNRLTIG; SEQ ID NO: 5, including γ377-395), or IIPFXRLXI (SEQ ID NO: 38) peptides. The antibodies can bind any of these epitopes, others.

Isoforms and variants of fibrinogen/fibrin proteins can also be targeted by the antibodies described herein. Such isoforms and variants of fibrinogen/fibrin proteins can have sequences that have between 55-100%, including at least about 70%, 75%, 80%, 85%, 90%, 95%, 96%, 97%, 98%, or 99% sequence identity to any of the fibrinogen/fibrin (reference) sequences described herein.

For example, a human fibrinogen sequence with NCBI accession number AAB59530.1 has the following sequence (SEQ ID NO:7), highlighting the (QSGLYFIKPLKANQQFLVY; SEQ ID NO:4), and γ364-395 (DNGIIWATWKTRWYSMKKTTMKIIPFNRLTIG; SEQ ID NO:5) sequences.

| | |
|---|---|
| 1 | MSWSLHPRNL ILYFYALLFL SSTCVAYVAT RDNCCILDER |
| 41 | FGSYCPTTCG IADFLSTYQT KVDKDLQSLE DILHQVENKT |
| 81 | SEVKQLIKAI QLTYNPDESS KPNMIDAATL KSRIMLEEIM |
| 121 | KYEASILTHD SSIRYLQEIY NSNNQKIVNL KEKVAQLEAQ |
| 161 | CQEPCKDTVQ IHDITGKDCQ DIANKGAKQS GLYFIKPLKA |
| 201 | NQQFLVYCEI DGSGNGWTVF QKRLDGSVDF KKNWIQYKEG |
| 241 | FGHLSPTGTT EFWLGNEKIH LISTQSAIPY ALRVELEDWN |
| 281 | GRISTADYAM FKVGPEADKY RLTYAYFAGG DAGDAFDGFD |
| 321 | FGDDPSDKFF TSHNGMQFST WDNDNDKFEG NCAEQDGSGW |
| 361 | WMNKCHAGHL NGVYYQGGTY SKASTPNGYD NGIIWATWKT |
| 401 | RWYSMKKTTM KIIPENRLTI GEGQQHHLGG AKQVRPEHPA |
| 421 | ETEYDSLYPE DDL |

The SEQ ID NO:7 fibrinogen sequence has one amino acid difference compared to the fibrinogen sequence with SEQ ID NO:39.

Isoforms and variants of fibrinogen/fibrin proteins can have at least 55% sequence identity, preferably 60%, preferably 70%, preferably 80%, preferably at least 900%, preferably at least 95%, preferably at least 96%, preferably at least 97% sequence, preferably at least 98%, preferably at least 99% identity to a reference sequence over a specified comparison window. Optimal alignment may be ascertained or conducted using the homology alignment algorithm of Needleman and Wunsch, J. Mol. Biol. 48:443-53 (1970).

In one embodiment, the antibody or fragments thereof bind P2 peptide (YSMKETTMKIIPFNRLTIG; SEQ ID NO: 1) and/or one or more of the peptides in Table 1 (which provides a list of antibody G epitopes derived from a fibrinogen peptide array. 15mer peptide sequences that resulted in greater than 2500 relative light units (RLU) are shown with corresponding signal intensity:

SEQ ID NO

| | | | |
|---|---|---|---|
| 6 | FIBA 218-232 | FKSQLQKVPPEWKAL | 6881.00 |
| 40 | FIBA 507-521 | FPGFFSPMLGEFVSE | 3371.00 |
| 8 | FIBA 514 528 | FSPMLGEFVSETESR | 5033.67 |
| 9 | FIBA 602-616 | RGHAKSRPVRGIHTS | 2566.67 |
| 41 | FIBB 63-77 | GGCLHADPDLGVLCP | 2779.67 |
| 42 | FIBB 71-85 | DLGVLCPTGCQLQEA | 2546.00 |
| 43 | FIBB 107-121 | VSQTSSSSFQYMYLL | 2933.00 |
| 13 | FIBB 139-153 | VNEYSSELEKHQLYI | 6792.67 |
| 44 | FIBB 191-205 | EYCRTPCTVSCNIPV | 2788.00 |
| 45 | FIBB 235-249 | PYRVYCDMNTENGGW | 3609.00 |
| 46 | FIBB 258-262 | SVDFGRKWDPYKQGF | 6908.33 |
| 17 | FIBB 283-297 | KNYCGLPGEYWLGND | 8779.67 |
| 18 | FIBB 315-329 | EDWKGDKVKAHYGGF | 5065.67 |
| 19 | FIBB 319-333 | GDKVKAHYGGFTVQN | 2667.00 |
| 20 | FIBB 391-405 | RKQCSKEDGGGWWYN | 2979.33 |
| 21 | FIBB 395-409 | SKEDGGGWWYNRCHA | 3467.67 |
| 22 | FIBB 407-421 | CHAANPNGRYYWGGQ | 9203.00 |
| 23 | FIBB 415-429 | RYYWGGQYTWDMAKH | 12865.67 |
| 24 | FIBB 427-431 | AKHGTDDGVVWMNWK | 2635.33 |
| 25 | FIBG 7-21 | NCCILDERFGSYCPT | 5758.33 |
| 47 | FIBG 15-29 | FGSYCPTTCGIADFL | 3149.33 |
| 27 | FIBG 123-137 | NLKEKVAQLEAQCQE | 3128.50 |
| 48 | FIBG 202-216 | DFKKNWIQYKEGFGH | 9084.33 |
| 29 | FIBG 206-220 | NWIQYKEGFGHLSPT | 6578.00 |
| 30 | FIBG 214-228 | FGHLSPTGTTEFWLG | 8264.67 |
| 31 | FIBG 287-301 | GDAFDGFDFGDDPSD | 11360.33 |
| 32 | FIBG 303-317 | FFTSHNGMQFSTWDN | 6749.33 |
| 33 | FIBG 335-349 | WMNKCHAGHLNGVYY | 50000.00 |

And/or one more peptides in Table 2 (which provides list of antibody G epitopes identified by a fibrinogen peptide array):

| Peptide ID (15mer) | 15mer AA Sequence |
|---|---|
| FIBA 218-232 (SEQ ID NO: 6) | FKSQLQKVPPEWKAL |
| FIBA 510-524 (SEQ ID NO: 37) | FPGFFSPMLGEFVSE |
| FIBB 139-153 (SEQ ID NO: 13) | VNEYSSELEKHQLYI |
| FIBB 283-297 (SEQ ID NO: 17) | KNYCGLPGEYWLGND |
| FIBB 407-421 (SEQ ID NO: 22) | CHAANPNGRYYWGGQ |
| FIBG 7-21 (SEQ ID NO: 25) | NCCILDERFGSYCPT |
| FIBG 203-217 (SEQ ID NO: 36) | DFKKNWIQYKEGFGH |
| FIBG 287-301 (SEQ ID NO: 31) | GDAFDGFDFGDDPSD |
| FIBG 303-317 (SEQ ID NO: 32) | FFTSHNGMQFSTWDN |
| FIBG 331-345 (SEQ ID NO: 34) | GSGWWMNKCHAGHLN |
| FIBG 335-349 (SEQ ID NO: 35) | WMNKCHAGHLNGVYY |

In some embodiments, the antibody binds one or more of the above SEQ ID NOs: 1, 6, 13, 17, 22, 25, 31, 32, 34, 35, 36 and/or 37 in Tables 1 and/or 2.

### Treatment of CNS Cancer

Provided herein is the description of how depletion of the γ³⁷⁷⁻³⁹⁵ fibrin epitope protects from the growth of B-cell lymphoma in the brain. Anti-fibrin tumor therapy would have the following advantages compared to other immunotherapy for tumor treatment: a) the approach utilizes a novel mechanism of action; no fibrin-targeting therapies are currently available for tumor treatment; b) blocking fibrin's interaction with a specific receptor on tumor cells is a highly selective intervention, and thus less likely to produce toxicity and/or side effects, c) understanding the molecular mechanisms involved opens up potential new therapeutic avenues for treating other CNS brain tumors or injury, or diseases involving blood-brain barrier (BBB) disruption.

Fibrin therapeutics targeting the γ377-395 fibrin epitope (peptides, antibodies, or small molecules) or depleting fibrin in the CNS to target oncogenic functions of fibrin in brain and other tumors are provided herein.

Fibrin therapeutics can include defibrinogenating agents, such as ancrod, batroxobin, crotalase, and thrombin.

Fibrin therapeutics can also include antibodies directed against fibrin.

Antibodies

Anti-fibrin antibodies can be used to treat B-cell lymphoma.

Antibodies can be raised against various epitopes of the fibrinogen, fibrin, or a portion or epitope thereof. Some antibodies for fibrinogen are available commercially. However, the antibodies contemplated for treatment pursuant to the methods and compositions described herein are preferably human or humanized antibodies and are generally specific for their fibrinogen/fibrin protein targets.

For example, the fibrinogen peptide γ₃₇₇₋₃₉₅ is the binding site for the CD11b I-domain of complement receptor 3 (CR3) (also known as CD11b/CD18, Mac-1, α_{M}β₂) and is needed for fibrin-induced activation of microglia and macrophages. A sequence for the CD11b/CD18 (Mac-1) protein is available as accession number P11215-1 from the Uniprot database and shown below as SEQ ID NO: 49:

```
                10 20 30 40
MALRVLLLTA LTLCHGFNLD TENAMTFQEN ARGFGQSVVQ
                50 60 70 80
LQGSRVVVGA PQEIVAANQR GSLYQCDYST GSCEPIRLQV
                90 100 110 120
PVEAVNMSLG LSLAATTSPP QLLACGPTVH QTCSENTYVK
              130 140 150 160
GLCFLFGSNL RQQPQKFPEA LRGCPQEDSD IAFLIDGSGS
              170 180 190 200
IIPHDFRRMK EFVSTVMEQL KKSKTLFSLM QYSEEFRIHF
              210 220 230 240
TFKEFQNNPN PRSLVKPITQ LLGRTHTATG IRKVVRELFN
              250 260 270 280
ITNGARKNAF KILVVITDGE KFGDPLGYED VIPEADREGV
              290 300 310 320
IRYVIGVGDA FRSEKSRQEL NTIASKPPRD HVFQVNNFEA
              330 340 350 360
LKTIQNQLRE KIFAIEGTQT GSSSSFEHEM SQEGFSAAIT
              370 380 390 400
SNGPLLSTVG SYDWAGGVFL YTSKEKSTFI NMTRVDSDMN
              410 420 430 440
DAYLGYAAAI ILENRVQSLV LGAPRYQHIG LVAMFRQNTG
              450 460 470 480
MWESNANVKG TQIGAYFGAS LCSVDVDSNG STDLVLIGAP
              490 500 510 520
HYYEQTRGGQ VSVCPLPRGR ARWQCDAVLY GEQGQPWGRF
              530 540 550 560
GAALTVLGDV NGDKLTDVAI GAPGEEDNRG AVYLFHGTSG
              570 580 590 600
SGISPSHSQR IAGSKLSPRL QYFGQSLSGG QDLTMDGLVD
              610 620 630 640
LTVGAQGHVL LLRSQPVLRV KAIMEFNPRE VARNVFECND
              650 660 670 680
QVVKGKEAGE VRVCLHVQKS TRDRLREGQI QSVVTYDLAL
              690 700 710 720
DSGRPHSRAV FNETKNSTRR QTQVLGLTQT CETLKLQLPN
              730 740 750 760
CIEDPVSPIV LRLNFSLVGT PLSAFGNLRP VLAEDAQRLF
              770 780 790 800
TALFPFEKNC GNDNICQDDL SITFSFMSLD CLVVGGPREF
              810 820 830 840
NVTVTVRNDG EDSYRTQVTF FFPLDLSYRK VSTLQNQRSQ
              850 860 870 880
RSWRLACESA SSTEVSGALK STSCSINHPI FPENSEVTFN
              890 900 910 920
ITFDVDSKAS LGNKLLLKAN VTSENNMPRT NKTEFQLELP
              930 940 950 960
VKYAVYMVVT SHGVSTKYLN FTASENTSRV MQHQYQVSNL
              970 980 990 1000
GQRSLPISLV FLVPVRLNQT VIWDRPQVTF SENLSSTCHT
            1010 1020 1030 1040
KERLPSHSDF LAELRKAPVV NQSIAVCQRI QCDIPFFGIQ
            1050 1060 1070 1080
EEFNATLKGN LSFDWYIKTS HNHLLIVSTA EILENDSVFT
            1090 1100 1110 1120
LLPGQGAFVR SQTETKVEPF EVPNPLPLIV GSSVGGLLLL
            1130 1140 1150
ALITAALYKL GFFKRQYKDM MSEGGPPGAE PQ
```

Desirable anti-fibrin/anti-fibrinogen antibodies can block the binding of Mac-1 (CD11b/CD18) to fibrin or fibrinogen.

The antibodies may be monoclonal antibodies. Such antibodies may also be humanized or fully human monoclonal antibodies. The antibodies can exhibit one or more desirable functional properties, such as high affinity binding to fibrinogen or fibrin.

Methods and compositions described herein can include antibodies that bind fibrinogen or fibrin. The antibodies can also bind to a combination of antibodies that bind to fibrinogen or fibrin, or a combination where each antibody type can separately bind fibrinogen or fibrin.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., a peptide or domain of fibrinogen or fibrin). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds fibrinogen or fibrin is substantially free of antibodies that specifically bind antigens other than fibrinogen or fibrin). An isolated antibody that specifically binds fibrinogen or fibrin may, however, have cross-reactivity to other antigens, such as isoforms or related fibrinogen and fibrin proteins from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VL and VH regions of the recombinant antibodies are sequences that, while derived from and related to human germline VL and VH sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human fibrinogen or fibrin” is intended to refer to an antibody that binds to human fibrinogen or fibrin with a K_{D} of 1×10⁻⁷ M or less, more preferably 5×10⁻⁸ M or less, more preferably 1×10⁻⁸ M or less, more preferably 5×10⁻⁹ M or less, even more preferably between 1×10⁻⁸ M and 1×10⁻¹⁰ M or less.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to human fibrinogen or fibrin. Preferably, an antibody of the invention binds to fibrinogen or fibrin with high affinity, for example with a K_{D} of 1×10⁻⁷ M or less.

Assays to evaluate the binding ability of the antibodies to fibrinogen or fibrin can be used, including for example, ELISAs, Western blots and RIAs. The binding kinetics (e.g., binding affinity) of the antibodies also can be assessed by standard assays known in the art, such as by Biacore™. analysis.

When each of the subject antibodies can bind to fibrinogen or fibrin, the VL and VH sequences can be “mixed and matched” to create other binding molecules that bind to fibrinogen or fibrin. The binding properties of such “mixed and matched” antibodies can be tested using the binding assays described above and assessed in assays described in the examples. When VL and VH chains are mixed and matched, a VII sequence from a particular VH/VL pairing can be replaced with a structurally similar VH sequence. Likewise, preferably a VL sequence from a particular VH/VL pairing is replaced with a structurally similar VL sequence.

Accordingly, in one aspect, the invention provides an isolated monoclonal antibody, or antigen binding portion thereof comprising:
(a) a heavy chain variable region comprising an amino acid sequence; and
(b) a light chain variable region comprising an amino acid sequence;
wherein the antibody specifically binds fibrinogen or fibrin.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin alphaᵥbeta₃ antibodies using a heavy and light chain variable CDR3 domain. Hence, in some cases a mixed and matched antibody or a humanized antibody contains a CDR3 antigen binding domain that is specific for fibrinogen or fibrin.

In particular, the invention provides monoclonal antibodies that specifically bind the γ³⁷⁷⁻³⁹⁵ epitope of the fibrin and fibrinogen γC domain, or any of the Bβ119-129 (YLLKDLWQKRQ, SEQ ID NO:3), γ163-181 (QSGLYFIKPLKANQQFLVY; SEQ ID NO:4) and γ364-395 (DNGIIWATWKTRWYSMKKTTMKIIPFNRLTIG; SEQ ID NO:5) sites.

Various polynucleotide and polypeptide sequences related to the 5B8 antibody (anti-fibrin antibody 5B8 (Ryu et al. Nat Immunol 19: 1212-1223 (2018))) are described herein. These sequences include the 5B8 light chain amino acid sequence (SEQ ID NO: 50), shown below.

| | |
|---|---|
| 1 | TFDSPYQVRR MRFSAQLLGL LVLWIPGSTA DIVMTQAAFS |
| 41 | NPITLGTSAS MSCRSSKSLL HSSGITYLSW YLQKPGQSPQ |
| 81 | LLIYQMSNLA SGVPDRFSSS GSGTDFTLRI SRVEAEDVGV |
| 121 | YYCAQNLELP LTFGAGTKLE LKRADAAPTV SACTKGEF |

Three 5B8 antibody light chain CDR amino acid sequences (CDR-L1, CDR-L2, and CDR-L3), are shown below as SEQ ID NO:10, 11, and 12, respectively.

The CDR-L1 sequence (SEQ ID NO: 10) is
RSSKSLLHSSGITYLS.
The CDR-L2 sequence (SEQ ID NO: 11) is QMSNLAS.
The CDR-L3 sequence (SEQ ID NO: 12) is AQNLELPLT.

5B8 antibody heavy chain amino acid sequence is shown below as (SEQ ID NO: 51).

| | |
|---|---|
| 1 | NTAFAGFGRN MRSLFSLQLL STQDLAMGWS CIIVLLVSTA |
| 41 | TGVHSQVQLQ QPGAELVRPG TSVKLSCKAS GYTFTSYWIH |
| 81 | WVKQRPGQGL EWIGLIDPSD SYTNYNQKFR GKATLIVDTS |
| 121 | SSTAYMQLSS LTSEDSAVYY CASSDPTGCW GQGTTLTVSP |
| 161 | ASTTPP |

Three heavy chain CDR amino acid sequences (CDR-H1, CDR-H2, and CDR-H3), are shown below as SEQ ID NO:14, 15, and 16, respectively.

The CDR-H1 sequence (SEQ ID NO: 14) is GYTFTSYWIH.
The CDR-H2 sequence (SEQ ID NO: 15) is
LIDPSDSYTNYNQKFR.
The CDR-H3 sequence (SEQ ID NO: 16) is SDPTGC.

The 5B8 antibody light chain nucleotide sequence is shown below as SEQ ID NO:52.

| | |
|---|---|
| 1 | ACTTTTGACT CACCATATCA AGTTCGCAGA ATGAGGTTCT |
| 41 | CTGCTCAGCT TCTGGGGCTG CTTGTGCTCT GGATCCCTGG |
| 81 | ATCCACTGCA GATATTGTGA TGACGCAGGC TGCATTCTCC |
| 121 | AATCCAATCA CTCTTGGAAC ATCAGCTTCC ATGTCCTGCA |
| 161 | GGTCTAGTAA GAGTCTCCTA CATAGTAGTG GCATCACTTA |
| 201 | TTTGTCTTGG TATCTGCAGA AGCCAGGCCA GTCTCCTCAG |
| 241 | CTCCTGATTT ATCAGATGTC CAACCTTGCC TCAGGAGTCC |
| 281 | CAGACAGGTT CAGTAGCAGT GGGTCAGGAA CTGATTTCAC |
| 321 | ACTGAGAATT AGCCGAGTGG AGGCTGAGGA TGTGGGTGTT |
| 361 | TATTACTGTG CTCAAAATCT AGAACTTCCG CTCACGTTCG |
| 401 | GTGCTGGGAC CAAGCTGGAG CTGAAACGGG CTGATGCTGG |
| 441 | ACCAACTGTA TCCGCATGCA CCAAGGGCGA ATTC |

The 5B8 antibody heavy chain nucleotide sequence is shown below as SEQ ID NO: 53.

| | |
|---|---|
| 1 | GAACACTGCG TTTGCTGGCT TTGGAAGAAA CATGAGATCA |
| 41 | CTGTTCTCTC TACAGTTACT GAGCACACAG GACCTCGCCA |
| 81 | TGGGATGGAG CTGTATCATT GTCCTCTTGG TATCAACAGC |
| 121 | TACAGGTGTC CACTCCCAGG TCCAACTGCA GCAGCCTGGG |
| 161 | GCTGAGCTGG TGAGGCCTGG GACTTCAGTG AAGTTGTCCT |
| 201 | GCAAGGCTTC TGGCTACACC TTCACCAGCT ACTGGATACA |
| 241 | CTGGGTAAAG CAGAGGCCTG GACAAGGCCT TGAGTGGATC |
| 281 | GGACTGATTG ATCCTTCTGA TAGTTATACT AACTACAATC |
| 321 | AAAAGTTCAG GGGCAAGGCC ACATTGACTG TAGACACATC |
| 361 | CTCCAGCACA GCCTACATGC AGCTCAGCAG CCTGACATCT |
| 401 | GAGGACTCTG CGGTCTATTA CTGTGCAAGC TCCGATCCTA |
| 441 | CAGGCTGCTG GGGCCAAGGC ACCACTCTCA CAGTCTCCCC |
| 481 | AGCTAGCACA ACACCCCCA |

Nucleotide sequences of the three 5B8 antibody light chain CDRs (CDR-L1, CDR-L2, and CDR-L3), are shown below as SEQ ID NO: 54, 55, and 56, respectively.

The 5B8 antibody light chain CDR-L1 nucleotide
sequence is:
(SEQ ID NO: 54)
AGGTCTAGTA AGAGTCTCCT ACATAGTAGT GGCATCACTT
ATTTGTCT.
The 5B8 antibody light chain CDR-L2 nucleotide
sequence is:
(SEQ ID NO: 55)
CAGATGTCCA ACCTTGCCTC.
The 5B8 antibody light chain CDR-L3 nucleotide
sequence is:
(SEQ ID NO: 56)
GCTCAAAATC TAGAACTTCC GCTCACG.

Nucleotide sequences of the three 5B8 antibody heavy chain CDRs (CDR-H1, CDR-H2, and CDR-H3), are shown below as SEQ ID NO: 57, 58, and 59, respectively.

The 5B8 antibody heavy chain CDR-H1 nucleotide
sequence is:
(SEQ ID NO: 57)
GGCTACACCT TCACCAGCTA CTGGATACAC.
The 5B8 antibody heavy chain CDR-H2 nucleotide
sequence is:
(SEQ ID NO: 58)
CTGATTGATC CTTCTGATAG TTATACTAAC TACAATCAAA
AGTTCAGGGG C.
The 5B8 antibody heavy chain CDR-H3 nucleotide
sequence is:
(SEQ ID NO: 59)
TCCGATCCTA CAGGCTGC.

In some cases, the methods and compositions described herein can include the 5B8 antibody. In other cases, the methods and compositions described herein do not include the 5B8 antibody.

Various polynucleotide and polypeptide sequences related to the antibody G or 45-C1C3 antibody (anti-fibrin antibody G or 45-C1C3 (U.S. Pat. Appln Ser No. 63/399,752) are described herein. These sequences include those shown below.

Antibody 45-C1C3 is a human IgGk resulting from a single CSF plasma cell obtained from a human subject with a MS diagnosis. 45-C1C3-VH and 45-C1C3-VLk PCR products were separately cloned into eukaryotic expression plasmid containing the constant regions of human IgG1 H chain, and human kL chain as described in Tiller et al., 2008 (J of Immunological Methods 329 (2008) 112-124). After cloning and confirmation of cloned 45-C1C3-VH and 45-C1C3-VLk sequences, plasmid DNA was produced in E. coli and purified (sequences provided herein). Soluble 45-C1C3 antibody was produced by transient expression in 293-T human embryonic kidney fibroblasts following co-transfection with 45-C1C3-VH and 45-C1C3-VLk encoding plasmid DNA by calcium phosphate precipitation. Transfected cells were cultured in serum and Ig free D-MEM. Supernatants were collected after 8 days of culture and 45-C1C3 antibody was purified using protein G sepharose.

A light or heavy chain variable region of an antibody has four framework regions interrupted by three hypervariable regions, known as complementary determining regions (CDRs). CDRs determine the specificity of antigen binding. The heavy chain and light chain each have three CDRs, designated from the N terminus as CDR1, CDR2, and CDR3 with the four framework regions flanking these CDRs. The amino acid sequences of the framework region are highly conserved and CDRs can be transplanted into other antibodies. Therefore, a recombinant antibody can be produced by combining CDRs from one or more antibodies with the framework of one or more other antibodies.

Polypeptides/antibodies of the invention comprise full-length heavy chain variable regions, full-length light chain variable regions, binding fragments or variants thereof, and combinations thereof.

G Heavy Chain Variable Region (V-D-J)
Nucleotide Sequence:
(SEQ ID NO: 60; cDNA)
gaagtgcagctggtggagtctgggggaggcgcggtccaacctgggaggt
ccctgagactctcctgtgcagcctctggagtcagtttcagtaacattgg
catgcactgggtccgccaggctccaggcaaggggctggagtgggtggca
cttatatcatctgatggacgtcatacacactatgcagactccgtgaagg
gccgattcaccatctccagagacaattccgagaacacgctctatctaca
gatcaacggcctgagagctgacgacacggctgtttattactgtgcgaaa
ggccttgacactcgtgcccgttacatggggaaattttatacttttgact
actggggccagggaaccctggtcaccgtctcctca
Amino acid Sequence:
(SEQ ID NO: 26)
HYADSVKGRFTISRDNSENTLYLQINGLRADDTAVYYCAKGLDTRARYM
GKFYTFDYWGQGTLVTVSS
G Kappa Light Chain Variable Region (V-J)
Nucleotide Sequence:
(SEQ ID NO: 61; cDNA)
gacatccagttgacccagtctccatcctccctgtctgcatctgtaggag
acagagtcaccatcacttgtcgggcaagtcagaacattggcgtcaattt
aaattggtatcaacagagaccagggaaaggcccgaaactcctagtctct
tctacatttagtttgcaaagtggggtcccatcaaggtttagtgccagtg
gagctgggacaaatttcactctcaccatcagcagtctgcaacctgagga
ctatgtcacttactactgtcaacagagttacagtagtccattcacattc
ggccctgggaccaaagtggatatcaaa
Amino acid Sequence:
(SEQ ID NO: 28)
DIQLTQSPSSLSASVGDRVTITCRASQNIGVNLNWYQQRPGKGPKLLVS
STFSLQSGVPSRFSASGAGTNFTLTISSLQPEDYVTYYCQQSYSSPFTF
GPGTKVDIK

Techniques for determining CDRs are generally known in the art. In some embodiments, the CDRs can be determined by approaches based on cross-species sequence variability. In some embodiments, the CDRs can be determined by approaches based on crystallographic studies of antigen-antibody complexes. In addition, combinations of these approaches are sometimes used in the art to determine CDRs. In certain embodiments, CDRs can be determined using sequence-based prediction tools. Such tools are generally available in the art, e.g., the Loupe V(D)J Browser provided by 10× Genomics® (Pleasanton, Calif.). For instance, in one embodiment, the single cell TCR sequencing of epitope reactive T-cell population can be conducted using the 10× Genomics® platform. Then the sequence can be processed using the Loupe V(D)J Browser to identify the clonotypes, V(D)J genes, and the CDR motifs, etc. More detailed information about the Loupe V(D)J Browser is available over the world-wide-web at site: support.10×genomics.com/single-cell-vdj/software/visualization/latest/tutorial-clonotypes, which is herein incorporated by reference in its entirety.

In some cases, the methods and compositions described herein can include the antibody G or 45-C1C3 antibody. In other cases, the methods and compositions described herein do not include the antibody G or 45-C1C3 antibody.

The sequences provided herein, including the fibrin, fibrinogen, epitope and antibody sequences, are exemplary. Isoforms and variants of these sequences can also be used in the methods and compositions described herein.

For example, isoforms and variants of the proteins and nucleic acids described herein, including the antibody sequences, can be used in the methods and compositions described herein so long as they are substantially identical to the fibrin and antibody sequences described herein. The terms “substantially identity” indicates that a polypeptide or nucleic acid has a sequence with between 55-100% sequence identity to a reference sequence, for example with at least 55% sequence identity, preferably 60%, preferably 70%, preferably 80%, preferably at least 90%, preferably at least 95%, preferably at least 96%, preferably at least 97% sequence, preferably at least 98%, preferably at least 99% identity to a reference sequence over a specified comparison window. Optimal alignment may be ascertained or conducted using the homology alignment algorithm of Needleman and Wunsch, J. Mol. Biol. 48:443-53 (1970).

An indication that two antibody or two polypeptide sequences are substantially identical is that both antibodies or both polypeptides have the same function, for example binding fibrin. The antibodies that are substantially identical to a 5B8 antibody sequence may not have exactly the same level of activity as the 5B8 antibody. Instead, the substantially identical antibody may exhibit greater or lesser levels of binding affinity to fibrin. For example, the substantially identical antibody or nucleic acid encoding the antibody may have at least about 40%, or at least about 50%, or at least about 60%, or at least about 70%, or at least about 80%, or at least about 90%, or at least about 95%, or at least about 97%, or at least about 98%, or at least about 100%, or at least about 105%, or at least about 110%, or at least about 120%, or at least about 130%, or at least about 140/0, or at least about 150%, or at least about 200% of the binding affinity of the 5B8 antibody described herein when measured by similar assay procedures.

### Screening Methods

Also described herein are screening methods that can be used to identify useful small molecules, polypeptides, and/or anti-fibrin antibodies. Such useful small molecules, polypeptides, and antibodies can be screened for binding fibrin, for inhibiting binding of Mac-1 and fibrin, or a combination thereof.

The methods can involve contacting a fibrin or fibrinogen with a test agent and detecting whether the test agent binds to the fibrin or fibrinogen. The methods can also involve detecting whether the test agent binds to a fibrinogen/fibrin peptide. The test agents, and therapeutic agents, can also bind combinations of these peptides.

In addition, the methods can involve detecting whether a test agent will compete with the 5B8 antibody for binding to fibrin or fibrinogen. The methods can also include detecting whether a test agent can inhibit the binding of Mac-1 with fibrin/fibrinogen. Such methods can also involve quantifying the affinity and/or specificity of binding to fibrin or fibrinogen.

Test agents that do bind to fibrin or fibrinogen can also be administered to an animal (e.g., an experimental animal or a model animal) with B-cell lymphoma and then determining whether the test agent can reduce or inhibit tumor size or otherwise treat B-cell lymphoma within the animal.

### Expression Systems

Nucleic acid segments encoding one or more anti-fibrin antibodies can be inserted into or employed with any suitable expression system. Commercially useful and/or therapeutically effective quantities of one or more anti-fibrin antibodies can also be generated from such expression systems.

Recombinant expression of nucleic acids is usefully accomplished using a vector, such as a plasmid. The vector can include a promoter operably linked to nucleic acid segment encoding one or more anti-fibrin antibodies or encoding one or antibody fragments.

The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes.

A variety of prokaryotic and eukaryotic expression vectors suitable for carrying, encoding and/or expressing anti-fibrin antibodies can be used. A variety of prokaryotic and eukaryotic expression vectors suitable for carrying, encoding and/or expressing anti-fibrin antibodies can be employed. Such expression vectors include, for example, pET, pET3d, pCR2.1, pBAD, pUC, and yeast vectors. The vectors can be used, for example, in a variety of in vivo and in vitro situations.

The expression cassette, expression vector, and sequences in the cassette or vector can be heterologous. As used herein, the term “heterologous” when used in reference to an expression cassette, expression vector, regulatory sequence, promoter, coding region, or nucleic acid refers to an expression cassette, expression vector, regulatory sequence, promoter, coding region, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA forms. Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleic acid segment encoding one or more anti-fibrin antibodies, or a fragment thereof

A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The expression of anti-fibrin antibodies or antibody fragments thereof from an expression cassette or expression vector can be controlled by any promoter capable of expression in prokaryotic cells or eukaryotic cells. Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV.

The expression cassette or vector can include nucleic acid sequence encoding a marker product. This marker product is used to determine if a vector or expression cassette encoding the anti-fibrin antibodies has been delivered to the cell and once delivered, is being expressed. Marker genes can include the E. coli lacZ gene which encodes β-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, the nucleic acid molecules, expression cassette and/or vectors encoding anti-fibrin antibodies can be introduced to a cell by any method including, but not limited to, calcium-mediated transformation, electroporation, microinjection, lipofection, particle bombardment and the like. The cells can also be expanded in culture and then administered to a subject, e.g., a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

In some cases, the transgenic cell can produce exosomes or microvesicles that contain nucleic acid molecules, expression cassettes and/or vectors encoding anti-fibrin antibodies, or a combination thereof. In some cases, the transgenic cell can produce exosomes or microvesicles that contain nucleic acid molecules that can target anti-fibrin antibodies to particular tissues. Microvesicles can mediate the secretion of a wide variety of proteins, lipids, mRNAs, and micro RNAs, interact with neighboring cells, and can thereby transmit signals, proteins, lipids, and nucleic acids from cell to cell (see, e.g., Shen et al., J Biol Chem. 286(16): 14383-14395 (2011); Hu et al., Frontiers in Genetics 3 (April 2012); Pegtel et al., Proc. Nat'l Acad Sci 107(14): 6328-6333 (2010); WO/2013/084000; each of which is incorporated herein by reference in its entirety. Cells producing such microvesicles can be used to express the anti-fibrin antibodies.

Transgenic vectors or cells with a heterologous expression cassette or expression vector can express the encoded antibodies or fragments thereof. Any of these vectors or cells can be administered to a subject. Exosomes produced by transgenic cells can also be used to administer anti-fibrin antibody-encoding nucleic acids or antibody fragment-encoding nucleic acids to the subject.

Methods and compositions that include antibodies can involve use of one or more types of anti-fibrin antibodies, one or more antibody fragments thereof, or a combination thereof.

### Compositions

The invention also relates to compositions containing the active agents described herein. Such active agents can antibodies, nucleic acids encoding antibodies (e.g., within an expression cassette or expression vector), polypeptides, small molecules, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the compositions can include antibody, polypeptide, or small molecule that can bind to a fibrinogen/fibrin peptide, including but not limited, to the γ³⁷⁷⁻³⁹⁵ fibrin epitope. In other embodiments, the compositions can include at least one nucleic acid or expression cassette encoding an antibody or polypeptide that can bind to a fibrinogen/fibrin peptide, including but not limited, to the γ³⁷⁷⁻³⁹⁵ fibrin epitope.

In some embodiments, the active agents of the invention (e.g., antibodies, nucleic acids encoding one or more antibody type (e.g., within an expression cassette or expression vector), polypeptides, small molecules, or a combination thereof), are administered in a “therapeutically effective amount.” Such a therapeutically effective amount is an amount sufficient to obtain the desired physiological effect.

To achieve the desired effect(s), the active agents may be administered as single or divided dosages. For example, active agents can be administered in dosages of at least about 0.01 mg/kg to about 500 to 750 mg/kg of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of antibodies, polypeptides, small molecules, or nucleic acid chosen for administration, the severity of the condition, the weight, the physical condition, the health, and the age of the mammal. Such factors can be readily determined by the clinician employing animal models or other test systems that are available in the art.

Administration of the active agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the active agents and compositions of the invention may be essentially continuous over a preselected period of time or may be in a series of spaced doses. Both local and systemic administration is contemplated. Local administration can be to the brain.

To prepare the antibodies, polypeptides, small molecules, nucleic acids, expression cassettes, and other agents are synthesized or otherwise obtained, purified as necessary or desired. These antibodies, polypeptides, small molecules, nucleic acids, expression cassettes, and other agents can be suspended in a pharmaceutically acceptable carrier and/or lyophilized or otherwise stabilized. The antibodies, polypeptides, small molecules, nucleic acids, expression cassettes, other agents, and combinations thereof can be adjusted to an appropriate concentration, and optionally combined with other desired agents. The absolute weight of a given antibody, polypeptide, small molecule nucleic acid, expression vector, and/or another agent included in a unit dose can vary widely. For example, about 0.01 to about 2 g, or about 0.1 to about 500 mg, of at least one antibody, nucleic acid, polypeptide, small molecule, expression cassette, and/or other agent, or a plurality of antibodies, nucleic acids, polypeptides, small molecules, expression cassettes, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of the agent for use in treatment will vary not only with the particular carrier selected but also with the route of administration, the severity of the condition being treated and the age and condition of the patient. Ultimately the attendant health care provider can determine proper dosage. In addition, a pharmaceutical composition can be formulated as a single unit dosage form.

Thus, one or more suitable unit dosage forms comprising the agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods available in the pharmaceutical arts. Such methods may include the step of mixing the agents with liquid carriers, solid matrices, semi-solid carriers, finely divided solid carriers or combinations thereof, and then, if necessary, introducing or shaping the product into the desired delivery system. For example, the agent(s) can be linked to a convenient carrier such as a nanoparticle, albumin, polyalkylene glycol, or be supplied in prodrug form. The agent(s), and combinations thereof, can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of active agents can also involve parenteral or local administration of the in an aqueous solution or sustained release vehicle.

Thus, while the agents can sometimes be administered in an oral dosage form, that oral dosage form can be formulated so as to protect the antibodies, polypeptides, small molecules, nucleic acids, expression cassettes, and combinations thereof from degradation or breakdown before the antibodies, polypeptides, small molecules, nucleic acids encoding such polypeptides/antibodies, and combinations thereof provide therapeutic utility. For example, in some cases the antibodies, polypeptides, small molecules, nucleic acids encoding such antibodies/polypeptides, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The agents can be formulated in dry form (e.g., in freeze-dried form), in the presence or absence of a carrier. If a carrier is desired, the carrier can be included in the pharmaceutical formulation, or can be separately packaged in a separate container, for addition to the agents, after packaging in dry form, in suspension, or in soluble concentrated form in a convenient liquid.

Active agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as anti-viral agents, antibacterial agents, antimicrobial agents, immune modulators, other monoclonal antibodies, blood thinners, and/or preservatives.

The present description is further illustrated by the following examples, which should not be construed as limiting in any way. The contents of all cited references (including literature references, issued patents, published patent applications as cited throughout this application) are hereby expressly incorporated by reference.

### EXAMPLES

### Example 1

This Example describes some of the materials and methods used in the development of the invention.

### Introduction

Central nervous system (CNS) lymphoma is a form of non-Hodgkin B-cell neoplasm that can occur as either a primary or a metastatic disease (1-3). Both primary and metastatic CNS lymphomas show accumulation of tumor cells around blood vessels, implicating the cerebrovasculature in their pathogenesis (2) Dissemination of lymphoma cells within the CNS is associated with poor prognosis. During CNS tumor development, the blood-brain barrier (BBB) can be damaged as a primary or a secondary event (4,5). Loss of integrity in the BBB closely correlates with how fast individual metastases grow (6), and meta-analyses indicate a significant association between BBB leakage and degree of malignancy (7,8). However, the relationship between BBB leakage and CNS lymphoma onset or progression has not been demonstrated. BBB disruption allows plasma protein extravasation and deposition in the brain parenchyma. Indeed, a large-scale proteomic profiling of the cerebrospinal fluid (CSF) of patients with CNS lymphoma identified increased levels of coagulation factors, including fibrinogen (9), which correlated with tumor grade and poor clinical outcome (10). Interestingly, activation of the coagulation cascade seems to play a role in tumor growth and metastasis-associated events (11). In addition, apart from its role in coagulation, fibrinogen has been shown to drive tissue inflammation during CNS injury and disease (12). However, whether fibrinogen is deposited in CNS tumors in lymphoma patients and its potential role in lymphoma accumulation in CNS tumors have not been investigated.

Provided herein is the use of human tumor samples and a mouse model of CNS lymphoma to demonstrate that CSF protein extravasation through a permeable BBB can alter the tumor microenvironment to regulate the CNS tropism behavior and adhesion properties of CNS tumors. The results demonstrate that fibrinogen deposition occurs in the CNS parenchyma of an orthotopic mouse model of human B-cell lymphoma and in primary CNS lymphoma patients. Unbiased proteomic profiling of CSF from patients with metastatic B-cell CNS lymphoma revealed a molecular link between coagulation and signaling related to CNS lymphoma growth and progression. Lymphoma cells adhered to fibrinogen in vitro, and two-photon imaging of lymphoma in the brain showed that lymphoma tends to cluster around sites of BBB disruption. These data show, for the first time, the cellular and molecular interplay of fibrinogen and lymphoma growth signaling pathways and provide tumor-specific targets for pharmacologic intervention in lymphoma associated with BBB disruption.

### Materials and Methods

### Cell Lines

Raji, Ramos, and Ly3 cells were obtained from ATCC (Manassas, VA). Lymphoma cells were cultured in high glucose Dulbecco's modified Eagle's medium supplemented with 10% fetal calf serum, 1% penicillin-streptomycin, and 1% nonessential amino acids. To keep genetic drift to a minimum, cells were maintained in culture for a maximum duration of 2 to 3 months after thawing.

### Mice

Athymic male mice (nu/nu; Simonsen Laboratories, Gilroy, CA) between the age of 4 and 6 weeks were used for tumor implantation experiments. Mice were housed under Institutional Animal Care and Use Committee guidelines in a temperature- and humidity-controlled facility with a 12-hour light-12-hour dark cycle and ad libitum feeding. All animal protocols were approved by the Committee on Animal Research at the University of California, San Francisco, and were in accordance with the NIH guidelines.

### Intracerebral Injection of Lymphoma Cells

Raji cells were modified to stably express firefly luciferase by lentiviral transduction, as described (13). Cultured cells were washed three times with phosphate-buffered saline (PBS), counted, and maintained on ice in Dulbecco's modified Eagle's medium until injection. Athymic mice were anesthetized with a mixture of ketamine/xylazine and placed in a stereotactic frame. Raji cells (5×10⁵ cells in 5 mL serum-free medium) were slowly injected at a rate of 1 mL/minute with a Hamilton syringe attached to a 26-gauge needle into the brain at the following coordinates: anteroposterior, 0.5 mm; mediolateral, −2.5 mm; and dorsoventral, −3 mm from the bregma.

### Functional Enrichment and Protein Network Analysis

Network visualization was performed in Cytoscape version 3.7.2 (14) with the plug-in for STRING Database. (15) In brief, proteome data on differentially expressed proteins (false discovery rate (FDR), <0.1) that were increased in CSF from 31 patients with brain lymphoma compared with nonneoplastic control conditions (9) were used as input (data not shown) and queried in STRING Human Protein Database with default parameters. A total of 75 differentially expressed proteins were clustered on the basis of STRING functional enrichment analysis for Reactome pathways and ranked by FDR P<0.05 and >10 enriched proteins per Reactome pathway term.

### Immunohistochemistry

Mice were transcardially perfused with PBS and 4% paraformaldehyde under anesthesia, and brains were frozen in OCT compound (Tissue-Tek, Torrance, CA), then cut into 10-mm cryosections. For immunohistochemistry, sections were permeabilized in 0.1% Triton X-100, blocked with 5% normal donkey serum, and incubated for 24 hours at 4° C. with primary antibodies against fibrinogen (1:1000), laminin (1:100; Sigma-Aldrich, St. Louis, MO), CD20 (1:500. BioLegend, San Diego, CA), CD11b (1:100; Abcam, Cambridge, MA), ionized calcium-binding adaptor protein-1 (Iba-1; 1:1000; Wako, Richmond, VA), or CD31 (1:200; Abcam). Sections were rinsed in PBS with 0.1% Triton X-100 and incubated with secondary antibodies conjugated with Alexa Fluor 488 or 594 (1:200; Jackson Immunochemicals, West Grove, PA) for 1 hour in the dark. After washing in PBS, sections were cover slipped with Prolong Gold antifading agent (Invitrogen, Carlsbad, CA). For diffuse large B-cell lymphoma and mucosa-associated lymphoma tissue array (US Biomax, Inc., Derwood, MD), deparaffinized sections were immunostained with fibrinogen antibody (1:1000). Images were acquired using an Axioplan II epifluorescence microscope (Zeiss, White Plains, NY) equipped with dry Plan-Neofluar objectives (10×0.3, 20×0.5, or 40×0.75 numerical aperture).

### Cranial Window Surgery

Mice were kept at 37° C. using a heating pad during all surgical and imaging procedures. Surgery was performed under ketamine/xylazine anesthesia (100/10 mg/kg body weight). Following shaving of the head, disinfection of the skin, and local s.c. injection of 2% lidocaine, an incision was made to expose the skull. The skull was thoroughly dried by removing all bone-attached membrane and fat tissue. A circular moat of about 3 to 4 mm in diameter was drilled over the primary hindlimb/forelimb somatosensory cortex immediately above the implanted Raji cells. A custom-designed (eMachineShop, Mahwah, NJ) steel head bar that included an imaging chamber was positioned over the craniotomy and was firmly affixed to the skull with cyanoacrylate glue and Metabond dental cement. A drop of prewarmed (37° C.) artificial CSF (HEPES based; 125 mmol/L NaCl, 10 mmol/L glucose, 10 mmol/L HEPES, 3.1 mmol/L CaCl₂, 2.7 mmol/L KCl, and 1.3 mmol/L MgCl₂; pH 7.4) was applied to the bone island. After 1 minute, the bone island was gently lifted off with a microscalpel blade (Nordland Blade number 6900; Salvin Dental, Charlotte, NC), followed by gentle flushing of the dura mater with prewarmed artificial CSF to clean and remove potential dural bleeding. A drop of artificial CSF was placed on top of the dura mater, and the craniotomy was closed and sealed using a round glass coverslip (4 mm in diameter, type 0; Warner Instruments, Holliston, MA), which was fixed to the skull and sealed with Flow-It ALC composite (Pentron, Orange, CA) cured under UV light.

### In Vivo Two-Photon Brain Microscopy

An Ultima IV two-photon microscope (Prairie Technologies/Bruker, Billerica, MA), equipped with a Mai Tai eHP DeepSee (Spectra-Physics/Newport, Santa Clara, CA) and an Insight X3 Ti:Sapphire femtosecond laser (Spectra-Physics/Newport; pulse width, <120 femtoseconds; tuning range, 690 to 1040 nm (Mai Tai) and 680 to 1300 nm (Insight X3); repetition rate, 80 MHz; Spectra-Physics/Newport), was used and tuned to an excitation wavelength of 950 nm (Mai Tai) and 1200 nm (Insight X3). Following retro-orbital injection of 100 mL of 10-kDa Alexa 647elabeled dextran dissolved in artificial CSF, imaging was performed 50 to 100 mm below the dura mater in the somatosensory cortex for vascular and tumor cell imaging. A 25×1.05 numerical aperture water-immersion lens (Olympus, CenterValley, PA) was used. Images were acquired in galvo scan mode at 512×512 pixels, 1.5 Hz, and a 1.0-mm z-step. The maximum laser power exiting the objective was <40 mW. An infrared-blocking filter and 560-nm dichroic were placed in the primary emission beam path before the nond-scanned detectors. A 660-nm dichroic and a 692/24-nm+607/45-nm bandpass filter were used to separate Alexa 647-labeled dextran and red fluorescent protein emissions, respectively; a 520-nm dichroic and a 542/27-nm+494/41-nm bandpass filter were used to separate yellow fluorescent protein and green fluorescent protein fluorescence emissions, respectively. A 45-minute time-lapse series, visualizing microglial morphology, Raji cell dynamics, and BBB leakage within a 60- to 70-mm Z-stack, was acquired in 3-minute intervals. As described (16,17), all imaging was performed at least 50 mm below dura to avoid any potential effects of the cranial window surgery on cellular activity, survival, and BBB integrity.

### Processing and Analysis of In Vivo Imaging Data

Three-dimensional reconstruction and volume rendering were performed with the three-dimensional Viewer plug-in within ImageJ software version 1.51 (NIH, Bethesda, MD; imagej.nih.gov/ij). Images were adjusted for brightness/contrast and background noise with ImageJ software using the Subtract Background and/or Remove Outliers plug-ins. For figures, two-dimensional representations of volumes were generated via maximum intensity projections along the z axis using ImageJ software version 1.51. Time-lapse movies were generated using maximum intensity z-projections of stacks acquired sequentially over time. If drift occurred over the acquisition period, z shifts were corrected manually by taking substacks represented at all time points and lateral shifts were corrected with the StackReg plug-ins. Parenchymal movement tracking of individual Raji cells in the in vivo two-photon time-lapse Zstacks was accomplished by measuring the change in xy coordinates of the pixels representing the edge of each identified moving Raji cell from time 0 (start of time-lapse imaging) to time 45 (end of time-lapse imaging). Total cell movement was defined as the sum of the change in x and y pixel coordinates. Tracking was performed in ImageJ software. Areas of BBB leakage were defined by the presence of intravenously injected fluorescent Alexa 647elabeled 70-kDa dextran in the brain parenchyma, as previously described (16). For each time lapse interval, the presence or absence of parenchymal Alexa 647-labeled dextran was assessed by measuring the Alexa 647 fluorescence signal histogram in all extravascular/parenchymal space regions, as observed in the entire field of view. Only fields of view showing 0% Alexa 647 fluorescence signal in all extravascular/parenchymal space regions and at all imaging time points were defined as areas without BBB leakage.

### Cell Adhesion Assay

Blood was collected from the heart of anesthetized wild-type or Fga^{-/-} mice into one-tenth volume of sodium citrate (Sigma-Aldrich). Plasma was prepared by centrifugation at 2500×g for 10 minutes at room temperature. Lymphoma cells were incubated in PBS containing 0.1 mmol/L Calcein-AM (Invitrogen) for 20 minutes and 100,000 cells per well were plated on 96-well black p-clear-bottomed microtiter plates (Greiner Bio-One, Monroe, NC) precoated with plasma. Cells were incubated on plasma for 2 hours. The nonadherent cells were washed off with PBS, and Calcein-AM fluorescence was detected at 488 nm/520 nm using a SpectraMax M5 microplate reader (Molecular Devices, San Jose, CA).

### Statistical Analysis

Data are presented as means t SEM. Statistical calculations were performed with GraphPad Prism version 6.03 (GraphPad Software, San Jose, CA). Statistical significance was determined with a nonparametric two-sided U-test or two-way analysis of variance, followed by the Tukey posttest (multiple comparisons). All animals survived to the end of the study, and all data points were included in analysis.

All histopathologic analysis and quantification were performed blinded to experimental groups.

### Results

### Biological Categorization of Significantly Altered Proteins in CNS Lymphoma Patients

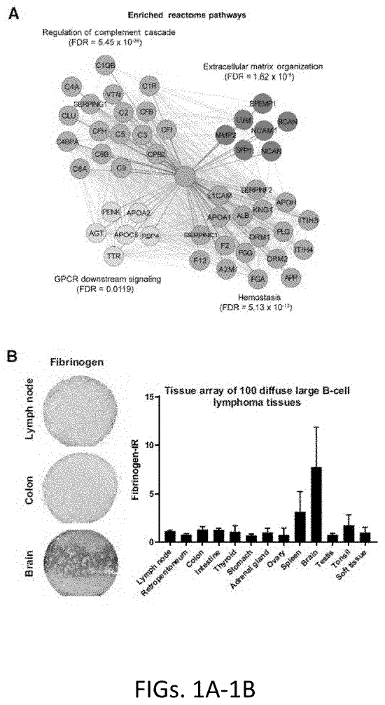

The proteome of the CSF of CNS lymphoma patients has been shown to differ significantly from that of control CSF (9). Although analysis of CSF proteins provides potential diagnostic and prognostic biomarker information, the above study did not reveal how the proteins found in the CSF may interact to promote CNS lymphoma. To fill this gap, CSF proteome data were subjected to a STRING functional enrichment analysis to rank Reactome pathway terms (FIG. 1A and data not shown). This analysis reveals functional links between proteins that may contribute jointly to a specific biological function. Because several annotations are branched together, the analysis was visualized as an enrichment network, which algorithmically clustered Reactome pathway terms with highly similar content, using the enrichment map plug-in in the Cytoscape environment. This revealed the highly connected pathways regulation of complement cascade (FDR=5.45×10⁻²⁶), extracellular matrix organization (FDR=1.62×10⁻⁹), G protein-coupled receptors downstream signaling (FDR=0.0119), and hemostasis (FDR=5.13×10⁻¹³). These data identified 37 coagulation proteins (hemostasis and regulation of complement cascade) that are significantly deregulated in CSF of CNS lymphoma patients and connected with tumor-associated biological signaling, increased extracellular matrix deposition, and G protein-coupled receptors signaling, and support a role for BBB disruption as a primary driver of lymphoma growth and progression.

Fibrinogen Deposits are Abundant in Brain Specimens from Patients with B-Cell Lymphoma

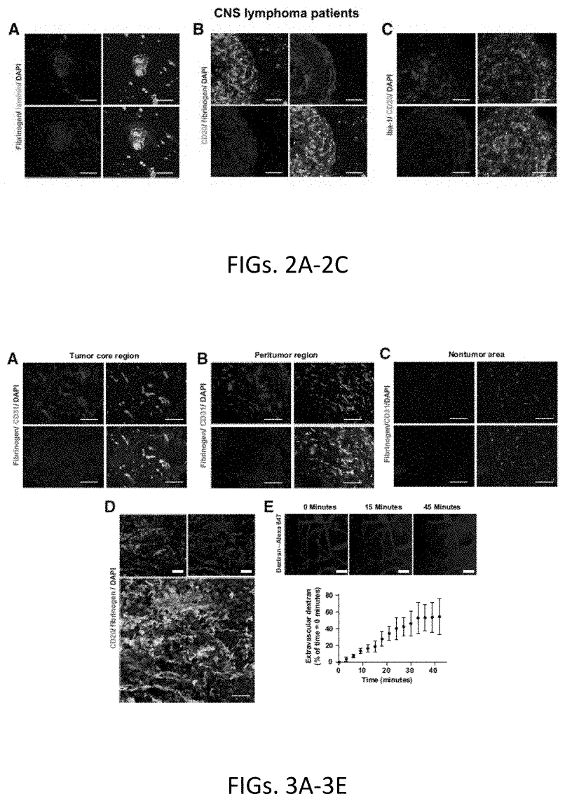

To determine whether fibrinogen forms deposits in B-cell lymphoma, immunohistochemical staining was performed on tissue arrays containing the malignant cores of diffuse large B-cell lymphoma (n=72) and of mucosa-associated lymphoma (n=28) in specified tissue specimens. Most CNS lymphoma specimens exhibited moderate to high levels of fibrinogen deposits, whereas lymph node and colon tissues had no observable immunoreactivity (FIG. 1B). Given the reported correlation between BBB leakage and poor prognosis for CNS lymphoma patients (7,8) presence of accumulated fibrinogen was examined in the brain of primary CNS lymphoma patients. To further characterize the localization of the fibrinogen deposits, immunostaining with an anti-pan laminin antibody was performed to detect the inner (endothelial) and outer (parenchymal) basement membrane. Fibrinogen was detected in the CNS parenchyma, including as extravascular deposits, as shown by double immunostaining with laminin (FIG. 2A). Perivascular fibrinogen deposits were found close to CD20⁺ lymphoma cells, suggesting the formation of a perivascular cuffing pattern, with lymphoma cells clustering around leaky blood vessels (FIG. 2B). Perivascular B-cell lymphoma cells were also in close proximity to Iba-1⁺ macrophage- or microglia-like cells (FIG. 2C). Thus, perivascular fibrin deposition may be a key spatial determinant of perivascular B-cell lymphoma clustering and microglia or macrophage accumulation in primary CNS lymphoma.

Perivascular Lymphomas Cluster Near Fibrinogen Deposits in Mouse Brains Injected with Cells from Patients with Primary CNS Lymphoma

To investigate the spatial correlation of BBB disruption with lymphoma growth, an orthotopic xenograft model of human CNS B-cell lymphoma in mice was used. Lymphoma cells from primary CNS lymphoma patients were stereotaxically injected into the cortex of Rag2^{-/-}γc^{-/-} mice, which lack B, T, and natural killer cells (FIG. 3) (18,19). Extravascular fibrinogen in the brain was assessed by the presence of fibrinogen immunoreactive areas outside CD31⁺ blood vessels. At the peak of the disease, day 14 after tumor transplantation, immunofluorescence staining revealed massive extravascular fibrinogen deposits around CD31⁺ blood vessels in the core and periphery of the lymphoma (FIGS. 3, A and B). As expected (20), CNS tumor blood vessels were structurally abnormal at the tumor-host interface and at central regions and differed from vessels in nontumor areas (FIG. 3, A-C). Areas with no tumor had no observable extravascular fibrinogen immunoreactivity (FIG. 3C). Double staining for CD20 and fibrinogen showed lymphoma cells clustered around blood vessel leaks stained with fibrinogen (FIG. 3D), suggesting that the injected lymphoma cells may adhere to fibrinogen deposits. Previous studies reported a direct correlation between tumor size and increased capillary permeability at the late stage of tumor progression, and only minimal contribution of the implant procedure to BBB permeability (21,22). To determine whether malignant lymphoma cells cause BBB leaks, real time in vivo tracking of active BBB leakage was performed in live mice injected intracerebrally with human B-cell lymphoma Raji cells (FIG. 3E). Imaging started immediately after a bolus i.v. injection of a fluorescently labeled dextran solution (blue). Parenchymal tumor clusters correlated with sites of active BBB disruption (FIG. 3E). These results identify active BBB leaks at sites of B-cell lymphoma in the CNS.

### Fibrinogen Induces Lymphoma Cell Adhesion

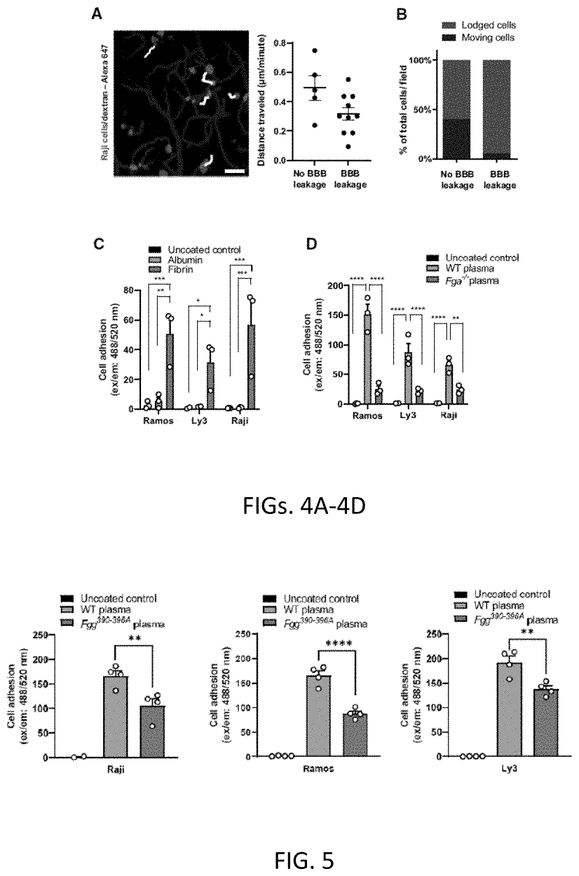

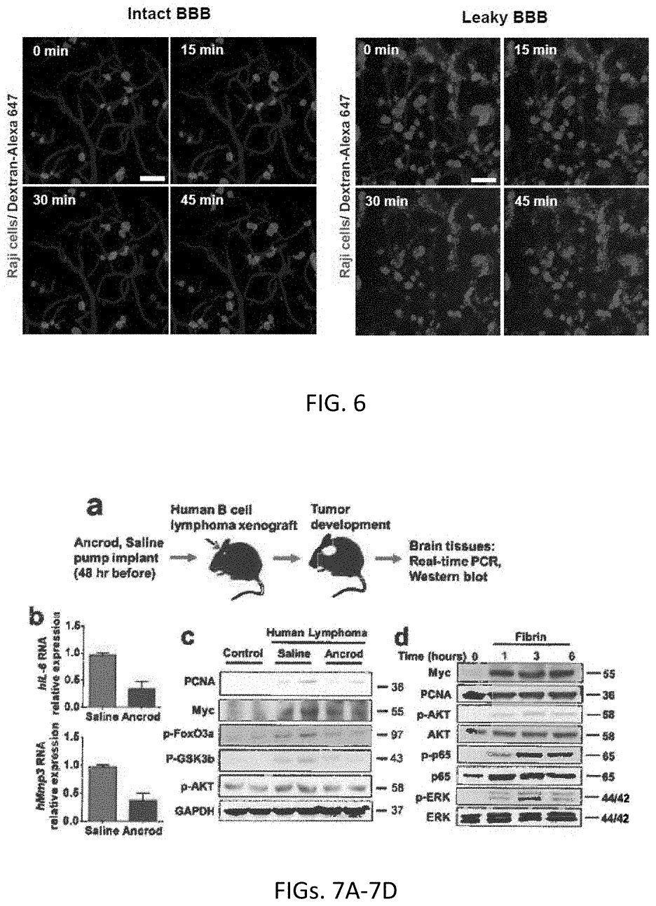

Over time, intravascular fluorescent dextran gradually leaked into the perivascular tissue that was occupied by lymphoma cells (FIG. 6). Time-lapse imaging and cell tracking showed that perivascular tumor cells at the site of BBB leakage exhibited reduced cell motility compared with cells distal to the BBB leakage areas (FIG. 4A). Furthermore, lymphoma cells at leakage sites were mostly lodged (94%), whereas cells at adjacent perivascular areas without leakage were either lodged (60%) or moving (40%) (FIG. 4B). In cell adhesion assays, fibrinogen coating induced significantly more adhesion of lymphoma cells than the blood protein albumin or an uncoated surface (FIG. 4C). Coating with wild-type plasma also induced lymphoma adhesion. In contrast, lymphoma adhesion was markedly lower with plasma from Fga^{-/-} mice, which contain virtually every plasma protein with the exception of fibrinogen (FIG. 4D). Overall, these results suggest that fibrinogen is a major plasma protein facilitating adhesion of lymphoma cells at sites of BBB disruption.

### Discussion

Provided herein is the first demonstration that fibrinogen deposits associated with lymphoma accumulation are a component of perivascular cuffing in CNS lymphoma patients. Reactome pathway analysis revealed that functional links between coagulation and hemostasis proteins contribute jointly to a specific functional CNS signaling pathway associated with lymphoma progression in CNS lymphoma patients. Using in vivo two-photon imaging in a mouse model of CNS lymphoma, lymphoma clusters were identified early, which increased with BBB disruption. These results further show that extravasated fibrinogen may act to retain CNS lymphoma cells near sites of BBB leakage by increasing cancer cell adhesion.

The formation of CNS lymphoma around sites of fibrin deposition suggests that activation of the coagulation cascade plays a role in tumor growth and metastasis associated events. However, the role of fibrinogen is not restricted to blood clotting, as components of the coagulation cascade within solid tissues (23,24) convert soluble fibrinogen into insoluble fibrin, which provides a provisional adhesive matrix for tumor cells (25-27). Furthermore, previous studies have identified fibrinogen as a molecular link between BBB disruption and neuroinflammation, as fibrinogen interacts with the CD11b/CD18 integrins expressed on microglia and infiltrating macrophages, activating proinflammatory signaling (16, 28-30). Thus, the fibrinogen deposits around BBB leaks in CNS lymphoma can mediate the local inflammatory response and affect disease progression.

Notably, the immunohistochemical methods used in this study do not discriminate between fibrinogen and fibrin. However, on activation of the coagulation cascade, conversion of fibrinogen to fibrin is associated with exposure of cryptic epitopes that transform fibrinogen from a blood factor to a potent activator of innate immunity, by allowing interaction with the CD11b integrin receptor (30,31) This can facilitate the clustering and subsequent growth progression of lymphoma cells.

There is a well-established link between tumor malignancy and altered hemostasis. Tumors rely heavily on blood supply for growth, and fibrin deposited within tumors is thought to promote angiogenesis by acting as a scaffold for vessel formation (32). The implication of fibrinogen in primary CNS lymphoma growth and adhesion is an important link in understanding the relationship between BBB disruption and lymphoma pathogenesis. Histologic evidence of fibrinogen accumulation in close association with lymphoma cell clustering in both a rodent model and human specimens suggests that fibrinogen can be a key factor in the bloodstream, promoting tumor progression. Intriguingly, analysis of plasma samples from Fga^{-/-} mice highlighted that fibrinogen is a major player among blood proteins in its ability to promote lymphoma adhesion. Taken together, the extent and nature of interaction of lymphoma with fibrinogen reported herein, and the early stage at which these interactions emerge, support the role of fibrinogen in the initial stages of disease pathogenesis. There may also be a role for fibrinogen in the later stages of tumor growth and a causal link with metastasis.

Given the potential role of blood coagulation factors in tumor progression, anticoagulants may represent a valid therapeutic approach. However, anticoagulants may interfere with coagulation in a non-specific manner, which could be detrimental to tumor patients. Furthermore, anticoagulants may affect the initial steps of tumor dissemination, such as tumor cell detachment from the extracellular matrix, invasion of surrounding tissues, and access to the systemic circulation (33). The current studies suggest that fibrinogen deposition is an important step of perivascular cuffing during the initial stages of CNS lymphoma formation, suggesting that targeting fibrin can influence lymphoma cell survival in a new microenvironment through interference with perivascular cuffing. The therapeutic potential of genetically or pharmacologically targeting the fibrinogen-CD11b interaction without compromising the coagulation properties of fibrinogen has been demonstrated in different animal models of CNS diseases (17,29,30,34). Similar approaches may be a promising strategy to target the early stages of CNS lymphomas.

### BIBLIOGRAPHY

1. Hochberg F H, Miller D C: Primary central nervous system lymphoma. J Neurosurg 1988, 68:835e853
2. Rubenstein J L, et al.: Gene expression and angiotropism in primary CNS lymphoma. Blood 2006, 107:3716e3723
3. Wang C C, Carnevale J, Rubenstein J L: Progress in central nervous system lymphomas. Br J Haematol 2014, 166:311e325
4. Dubois L G, et al.: Gliomas and the vascular fragility of the blood brain barrier. Front Cell Neurosci 2014, 8:418
5. Lee J, et al.: Glioma-induced remodeling of the neurovascular unit. Brain Res 2009, 1288:125e134
6. Osswald M, et al.: Impact of blood-brain barrier integrity on tumor growth and therapy response in brain metastases. Clin Cancer Res 2016, 22:6078e6087
7. Wang P F, et al.: Higher plasma fibrinogen levels are associated with malignant phenotype and worse survival in patients with glioblastomas. J Cancer 2018, 9:2024e2029
8. Perisanidis C, et al.: Prognostic role of pretreatment plasma fibrinogen in patients with solid tumors: a systematic review and meta-analysis. Cancer Treat Rev 2015, 41: 960e970
9. Roy S, et al.: Protein biomarker identification in the CSF of patients with CNS lymphoma. J Clin Oncol 2008, 26: 96e105
10. Troppan K T, et al.: The clinical significance of fibrinogen plasma levels in patients with diffuse large B cell lymphoma. J Clin Pathol 2016, 69:326e330
11. Francis J L, Biggerstaff J, Amirkhosravi A: Hemostasis and malignancy. Semin Thromb Hemost 1998, 24:93e109
12. Petersen M A, Ryu J K, Akassoglou K: Fibrinogen in neurological diseases: mechanisms, imaging and therapeutics. Nat Rev Neurosci 2018, 19:283e301
13. Kadoch C, et al.: Pathologic correlates of primary central nervous system lymphoma defined in an orthotopic xenograft model. Clin Cancer Res 2009, 15:1989e1997
14. Shannon P, et al.: Cytoscape: a software environment for integrated models of biomolecular interaction networks. Genome Res 2003, 13:2498e2504
15. Szklarczyk D, et al.: The STRING database in 2017: quality-controlled protein-protein association networks, made broadly accessible. Nucleic Acids Res 2017, 45:D362eD368
16. Davalos D, et al.: Fibrinogen-induced perivascular microglial clustering is required for the development of axonal damage in neuroinflammation. Nat Commun 2012, 3:1227
17. Merlini M, et al.: Fibrinogen induces microglia-mediated spine elimination and cognitive impairment in an Alzheimer's disease model. Neuron 2019, 101:1099e1108.e6
18. Rubenstein J L, et al.: CXCL13 plus interleukin 10 is highly specific for the diagnosis of CNS lymphoma. Blood 2013, 121:4740e4748
19. Geng H, et al.: Targeting NF-KB activation in novel intracranial models of CNS lymphoma. Blood 2016, 128:777
20. Nagy J A, Chang S H, Dvorak A M, Dvorak H F: Why are tumour blood vessels abnormal and why is it important to know? Br J Cancer 2009, 100:865e869
21. Hiesiger E M, et al.: Opening the blood-brain and blood-tumor barriers in experimental rat brain tumors: the effect of intracarotid hyperosmolar mannitol on capillary permeability and blood flow. Ann Neurol 1986, 19:50e59
22. Wiranowska M, et al.: Evaluation of blood-brain barrier permeability and the effect of interferon in mouse glioma model. J Neurooncol 1992, 14: 225e236
23. Furie B, Furie B C: The molecular basis of blood coagulation. Cell 1988, 53:505e518
24. Davalos D, Akassoglou K: Fibrinogen as a key regulator of inflammation in disease. Semin Immunopathol 2012, 34:43e62
25. Dvorak H F, et al.: Vascular permeability factor, fibrin, and the pathogenesis of tumor stroma formation. Ann N Y Acad Sci 1992, 667:101e11
26. Harris N L, et al.: Fibrin deposits in Hodgkin's disease. Am J Pathol 1982, 108:119e129
27. Bardos H, et al.: Fibrin deposition in primary and metastatic human brain tumours. Blood Coagul Fibrinolysis 1996, 7:536e548
28. Ryu J K, et al.: Blood coagulation protein fibrinogen promotes autoimmunity and demyelination via chemokine release and antigen presentation. Nat Commun 2015, 6:8164
29. Adams R A, et al.: The fibrin-derived gamma377-395 peptide inhibits microglia activation and suppresses relapsing paralysis in central nervous system autoimmune disease. J Exp Med 2007, 204:571e582
30. Ryu J K, et al.: Fibrin-targeting immunotherapy protects against neuroinflammation and neurodegeneration. Nat Immunol 2018, 19:1212e1223
31. Davalos D, et al.: Early detection of thrombin activity in neuroinflammatory disease. Ann Neurol 2014, 75: 303e308
32. Daly M E, et al.: Hemostatic regulators of tumor angiogenesis: a source of antiangiogenic agents for cancer treatment? J Natl Cancer Inst 2003, 95:1660e1673
33. Hejna M, Raderer M, Zielinski C C: Inhibition of metastases by anticoagulants. J Natl Cancer Inst 1999, 91:22e36
34. Akassoglou K: The immunology of blood: connecting the dots at the neurovascular interface. Nat Immunol 2020, 21: 710e712

### Example 2

### Design of Fibrin-Targeting Immunotherapy.

The C-terminus of the fibrinogen γ chain has two different sites at γ₄₀₀₋₄₁₁ and γ₃₇₇₋₃₉₅ that are involved in platelet engagement and inflammation respectively. These two sites are distinct domains within the three-dimensional structure of the fibrinogen protein. Peptide γ₄₀₀₋₄₁₁ is the binding site for the platelet α_{IIb}β₃ integrin receptor and is required for platelet aggregation. Peptide γ₃₇₇₋₃₉₅ is the binding site for the CD11b I-domain of complement receptor 3 (CR3) (also known as CD11b/CD18, Mac-1, α_{M}β₂; Ugarova et al. Biochemistry 42, 9365-9373 (2003)) and is required for fibrin-induced activation of microglia and macrophages. The γ₃₇₇₋₃₉₅ binding site is considered “cryptic” in the soluble fibrinogen molecule and is exposed only upon conversion of fibrinogen to insoluble fibrin.

The inventors had previously prepared antibodies against various fibrin epitopes using the following procedures.

Peptides corresponding to the exact amino acids on the γ chain of fibrin that have been shown to be needed for the interaction of fibrin/fibrinogen with Mac-1 were synthesized (Peptide #1: CGWTVLQKRIDGSL (SEQ ID NO: 62) and Peptide #2: CKKTTMKIIPFNRLTIG (SEQ ID NO: 63)). These two peptides were synthesized with N-terminal cysteine residues to allow for conjugation to the carrier protein keyhole limpet hemocyanin (KLH) which promotes a robust antibody response in vivo. Both peptides were used to immunize three mice generating an antibody response in these mice. Preliminary serum screening revealed a strong antibody titer against these peptides and lead to the subsequent generation of hybridomas producing clonal antibodies against these two peptide sequences. The initial screening of 480 hybridoma clones was performed by ELISA against both peptides as well as the carrier protein. The positive clones were expanded and retested to confirm peptide epitope reactivity by ELISA. The final results of this initial screen resulted in 46 clones that were specific to either Peptide #1 or #2. In depth analysis of these ELISA results identified 16 target candidates for further examination. These 16 clones were screened for their ability to block microglial adhesion via the Mac-1 receptor on full length fibrinogen. Tissue culture wells were coated with 50 μg/mL fibrinogen upon which microglia cells (200,000 cells/mL) were plated in the presence of these antibody clones. Wells were washed after 30 minutes and the remaining adherent cells were stained with 0.1% crystal violet. Stained cells were fixed with 1% PFA and solubilized with 0.5% Triton X-100. Five of these clones showed a significant ability, similar to that of a commercially available blocking antibody to Mac-1 (M1/70), to prevent microglial adhesion to fibrinogen as assessed by absorbance measurements at 595 nm.

Clones 1A5, 1D6 and 1E3 recognized the Peptide #1 epitope while clones 4E11 and 5B8 recognize the Peptide #2 epitope. The 5B8 monoclonal antibody has previously been shown by the inventors to inhibit neuroinflammation (Ryu et. Nat Immunol. 19(11): 1212-1223 (November 2018).

The five antibody preparations were further analyzed for their ability to recognize fibrinogen by western blot. All five antibodies recognized fibrinogen's γ chain to a similar degree. To examine whether these antibodies recognized fibrinogen in a dose dependent manner an ELISA was performed on full length coated fibrinogen. All five antibodies were found to specifically bind increasing concentrations of full-length fibrinogen. From these five antibodies three were chosen (I E3, 4E11 and 5B8, having greater than 50% inhibition of Mac-1 binding to the fibrin or fibrinogen γC domain when measured by shift in absorbance) for isolation and large-scale purification.

Antibodies 5B8, 4E11, and 4F1 had the highest selectivity and specificity for the γ₃₇₇₋₃₉₅ region of fibrinogen. All antibodies against cryptic epitopes bound with higher affinity to fibrin than to fibrinogen. Conversion of fibrinogen into fibrin exposes amino acids 377-395 in the fibrinogen chain. Hence, this region may be more accessible in fibrin than in fibrinogen. Among antibodies targeting γ₃₇₇₋₃₉₅, the 5B8 antibodies bound fibrin to the greatest degree with minimal binding to soluble fibrinogen. Competitive binding assays showed that 5B8 bound to human and mouse γ₃₇₇₋₃₉₅ peptides, but not γ₁₉₀₋₂₀₂ peptide. The 5B8 antibodies also inhibited binding of the CD11b I-domain to fibrin, indicating that the 5B8 antibodies interfere with the ligand-receptor interaction.

### Example 3

Fibrin upregulates oncogenic signaling in B-cell lymphoma in vivo and in vitro. It was examined whether fibrin is required for the activation of B-cell lymphoma in vivo. To determine the effects of fibrin on B-cell lymphoma, fibrinogen was pharmacologically depleted in mice with the fibrin-depleting agent ancrod. At 14 days after injecting B-cell lymphoma cells, the effects of fibrinogen on tumor cells were assessed by real time-PCR with human-specific primers (FIG. 7A). In ancrod-treated mice, expression of the pro-inflammatory cytokines MMP-3 and IL-6 was significantly less in injected tumor cells than in control mice that received saline (FIG. 7B). This suggests that extravasated fibrinogen induces activation of lymphoma cells. To determine the potential molecular mechanisms underlying this effect in vivo, several oncogenic signaling pathways that affect lymphoma were analyzed, including the proto-oncogene markers (Myc and BCL6), a cell proliferation marker (PCNA), PI3K signaling pathways (PI3K, AKT, Fox03a), Wnt signaling (GSK3b), and MAPK signaling pathways (ERK). The expression of these proteins was analyzed after injecting B-cell lymphoma into mice pharmacologically depleted of fibrinogen. By western blotting of brain samples, we found that the phosphorylation of Fox03a and GSK3b was dramatically less in ancrod-treated mice than saline-treated mice after injecting B-cell lymphoma cells (FIG. 7C). These results suggest that up-regulation of oncogenic signaling pathways is regulated by fibrinogen signaling in lymphoma, likely via its ability to confer a proliferative advantage to these cells. To determine if fibrin directly regulates a specific signaling pathway that affects lymphoma growth, the AKT, NF-KB, and ERK pathways, which regulate tumor-cell growth, survival, and proliferation, were examined. In the presence of fibrin, lymphoma cells showed dramatically more phosphorylated AKT, p6 5 subunit of the NF-KB complex, ERK, and rnyc expression than non-stimulated cells (FIG. 7D).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed is:

1. A method to treat or prevent cancer comprising administering anti-fibrin and/or anti-fibrinogen antibodies to a subject in need thereof.

2. The method of claim 1, wherein the cancer is a cancer of the central nervous system (CNS).

3. The method of claim 2, wherein the CNS cancer has blood brain barrier (BBB) disruption.

4. The method of claim 1, wherein there is malignant tissue in brain or spinal cord of the subject.

5. The method of claim 2, wherein the CNS cancer is a lymphoma.

6. The method of claim 5, wherein the lymphoma is a B-cell lymphoma.

7. The method of claim 2, wherein the CNS cancer is metastatic.

8. The method of claim 1, wherein the anti-fibrin and/or anti-fibrinogen antibodies are human antibodies, humanized antibodies or mouse antibodies.

9. The method of claim 1, wherein the anti-fibrin antibodies specifically bind to fibrin polypeptides or peptides thereof.

10. The method of claim 1, wherein the anti-fibrin antibodies bind to a fibrin domain or fragment.

11. The method of claim 1, wherein the anti-fibrin antibodies bind to a fibrin domain or fragment with higher affinity than fibrinogen.

12. The method of claim 1, wherein the anti-fibrin and/or anti-fibrinogen antibodies bind to an epitope of the fibrin or fibrinogen yC domain.

13. The method of claim 1, wherein the anti-fibrin and/or anti-fibrinogen antibodies bind to a peptide comprising one or more peptides in Table 1 and/or Table 2 and SEQ ID NO: 1 of human fibrin or fibrinogen yC domain.

14. The method of claim 1, wherein the anti-fibrin and/or anti-fibrinogen antibodies block binding of fibrin to Mac-1.

15. The method of claim 1, wherein the anti-fibrin and/or anti-fibrinogen antibodies block macrophage or microglia activation.

16. The method of claim 1, wherein the anti-fibrin antibodies and/or anti-fibrinogen antibodies comprise a CDR region with a sequence comprising SEQ ID NOs:10-12 and 14-16, or combination of CDR regions with sequences comprising SEQ ID NO:10, 11, 12, 14, 15, or 16.

17. The method of claim 1, wherein the anti-fibrin antibodies and/or anti-fibrinogen antibodies comprise a SEQ ID NOs: 26 and 28.

18. A method to block tumor cell interactions with fibrin comprising contacting one or more tumor cells with an anti-fibrin and/or anti-fibrinogen antibodies.

19. The method of claim 18, wherein the anti-fibrin and/or anti-fibrinogen antibodies are human antibodies, humanized antibodies or mouse antibodies.

20. The method of claim 19, wherein the anti-fibrin and/or anti-fibrinogen antibodies comprise a CDR region with a sequence comprising SEQ ID NOs:10-12 and 14-16, or combination of CDR regions with sequences comprising SEQ ID NO:10, 11, 12, 14, 15, or 16.

21. The method of claim 18, wherein the anti-fibrin and/or anti-fibrinogen antibodies comprise a SEQ ID NOs: 26 and 28.

22. A method of disrupting adhesion of lymphoma cells at a BBB disruption comprising contacting said cells with one or more anti-fibrin and/or anti-fibrinogen antibodies.

23. The method of claim 22, wherein the antibodies are human antibodies or humanized antibodies.

24. The method of claim 22, wherein the anti-fibrin and/or anti-fibrinogen antibodies comprise a CDR region with a sequence comprising SEQ ID NOs:10-12 and 14-16, or combination of CDR regions with sequences comprising SEQ ID NO:10, 11, 12, 14, 15, or 16.

25. The method of claim 22, wherein the anti-fibrin and/or anti-fibrinogen antibodies comprise a SEQ ID NOs: 26 and 28.


SEQUENCE LISTING (63 sequences)

SEQ ID NO: 1 - protein, 19 aa, Homo sapiens
YSMKETTMKIIPFNRLTIG

SEQ ID NO: 2 - protein, 16 aa, Homo sapiens
KKTTMKIIPFNRLTIG

SEQ ID NO: 3 - protein, 11 aa, Homo sapiens
YLLKDLWQKRQ

SEQ ID NO: 4 - protein, 19 aa, Homo sapiens
QSGLYFIKPLKANQQFLVY

SEQ ID NO: 5 - protein, 32 aa, Homo sapiens
DNGIIWATWKTRWYSMKKTTMKIIPFNRLTIG

SEQ ID NO: 6 - protein, 15 aa, Homo sapiens
FKSQLQKVPPEWKAL

SEQ ID NO: 7 - protein, 453 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRIMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 8 - protein, 15 aa, Homo sapiens
FSPMLGEFVSETESR

SEQ ID NO: 9 - protein, 15 aa, Homo sapiens
RGHAKSRPVRGIHTS

SEQ ID NO: 10 - protein, 16 aa, Homo sp.
RSSKSLLHSSGITYLS

SEQ ID NO: 11 - protein, 7 aa, Homo sapiens
QMSNLAS

SEQ ID NO: 12 - protein, 9 aa, Homo sapiens
AQNLELPLT

SEQ ID NO: 13 - protein, 15 aa, Homo sapiens
VNEYSSELEKHQLYI

SEQ ID NO: 14 - protein, 10 aa, Homo sapiens
GYTFTSYWIH

SEQ ID NO: 15 - protein, 16 aa, Homo sapiens
LIDPSDSYTNYNQKFR

SEQ ID NO: 16 - protein, 6 aa, Homo sapiens
SDPTGC

SEQ ID NO: 17 - protein, 15 aa, Homo sapiens
KNYCGLPGEYWLGND

SEQ ID NO: 18 - protein, 15 aa, Homo sapiens
EDWKGDKVKAHYGGF

SEQ ID NO: 19 - protein, 15 aa, Homo sapiens
GDKVKAHYGGFTVQN

SEQ ID NO: 20 - protein, 15 aa, Homo sapiens
RKQCSKEDGGGWWYN

SEQ ID NO: 21 - protein, 15 aa, Homo sapiens
SKEDGGGWWYNRCHA

SEQ ID NO: 22 - protein, 15 aa, Homo sapiens
CHAANPNGRYYWGGQ

SEQ ID NO: 23 - protein, 15 aa, Homo sapiens
RYYWGGQYTWDMAKH

SEQ ID NO: 24 - protein, 15 aa, Homo sapiens
AKHGTDDGVVWMNWK

SEQ ID NO: 25 - protein, 15 aa, Homo sapiens
NCCILDERFGSYCPT

SEQ ID NO: 26 - protein, 126 aa, Homo sapiens
EVQLVESGGGAVQPGRSLRLSCAASGVSFSNIGMHWVRQAPGKGLEWVALISSDGRHTHYADSVKGRFTISRDNSENTLYLQINGLRADDTAVYYCAKGLDTRARYMGKFYTFDYWGQGTLVTVSS

SEQ ID NO: 27 - protein, 15 aa, Homo sapiens
NLKEKVAQLEAQCQE

SEQ ID NO: 28 - protein, 107 aa, Homo sapiens
DIQLTQSPSSLSASVGDRVTITCRASQNIGVNLNWYQQRPGKGPKLLVSSTFSLQSGVPSRFSASGAGTNFTLTISSLQPEDYVTYYCQQSYSSPFTFGPGTKVDIK

SEQ ID NO: 29 - protein, 15 aa, Homo sapiens
NWIQYKEGFGHLSPT

SEQ ID NO: 30 - protein, 15 aa, Homo sapiens
FGHLSPTGTTEFWLG

SEQ ID NO: 31 - protein, 15 aa, Homo sapiens
GDAFDGFDFGDDPSD

SEQ ID NO: 32 - protein, 15 aa, Homo sapiens
FFTSHNGMQFSTWDN

SEQ ID NO: 33 - protein, 15 aa, Homo sapiens
WMNKCHAGHLNGVYY

SEQ ID NO: 34 - protein, 15 aa, Homo sapiens
GDGWWMNKCHAGHLN

SEQ ID NO: 35 - protein, 15 aa, Homo sapiens
WMNKCHAGHLNGVYY

SEQ ID NO: 36 - protein, 15 aa, Homo sapiens
DFKKNWIQYKEGFGH

SEQ ID NO: 37 - protein, 15 aa, Homo sapiens
FPGFFSPMLGEFVSE

SEQ ID NO: 38 - protein, 9 aa, Homo sapiens
IIPFXRLXI

SEQ ID NO: 39 - protein, 437 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 40 - protein, 15 aa, Homo sapiens
FPGFFSPMLGEFVSE

SEQ ID NO: 41 - protein, 15 aa, Homo sapiens
GGCLHADPDLGVLCP

SEQ ID NO: 42 - protein, 15 aa, Homo sapiens
DLGVLCPTGCQLQEA

SEQ ID NO: 43 - protein, 15 aa, Homo sapiens
VSQTSSSSFQYMYLL

SEQ ID NO: 44 - protein, 15 aa, Homo sapiens
EYCRTPCTVSCNIPV

SEQ ID NO: 45 - protein, 15 aa, Homo sapiens
PYRVYCDMNTENGGW

SEQ ID NO: 46 - protein, 15 aa, Homo sapiens
SVDFGRKWDPYKQGF

SEQ ID NO: 47 - protein, 15 aa, Homo sapiens
FGSYCPTTCGIADFL

SEQ ID NO: 48 - protein, 15 aa, Homo sapiens
DFKKNWIQYKEGFGH

SEQ ID NO: 49 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 50 - protein, 158 aa, Homo sapiens
TFDSPYQVRRMRFSAQLLGLLVLWIPGSTADIVMTQAAFSNPITLGTSASMSCRSSKSLLHSSGITYLSWYLQKPGQSPQLLIYQMSNLASGVPDRFSSSGSGTDFTLRISRVEAEDVGVYYCAQNLELPLTFGAGTKLELKRADAAPTVSACTKGEF

SEQ ID NO: 51 - protein, 166 aa, Homo sapiens
NTAFAGFGRNMRSLFSLQLLSTQDLAMGWSCIIVLLVSTATGVHSQVQLQQPGAELVRPGTSVKLSCKASGYTFTSYWIHWVKQRPGQGLEWIGLIDPSDSYTNYNQKFRGKATLTVDTSSSTAYMQLSSLTSEDSAVYYCASSDPTGCWGQGTTLTVSPASTTPP

SEQ ID NO: 52 - DNA, 474 bp, Homo sapiens
  source 1..474: mol_type = other DNA
acttttgactcaccatatcaagttcgcagaatgaggttctctgctcagcttctggggctgcttgtgctctggatccctggatccactgcagatattgtgatgacgcaggctgcattctccaatccaatcactcttggaacatcagcttccatgtcctgcaggtctagtaagagtctcctacatagtagtggcatcacttatttgtcttggtatctgcagaagccaggccagtctcctcagctcctgatttatcagatgtccaaccttgcctcaggagtcccagacaggttcagtagcagtgggtcaggaactgatttcacactgagaattagccgagtggaggctgaggatgtgggtgtttattactgtgctcaaaatctagaacttccgctcacgttcggtgctgggaccaagctggagctgaaacgggctgatgctgcaccaactgtatccgcatgcaccaagggcgaattc

SEQ ID NO: 53 - DNA, 499 bp, Homo sapiens
  source 1..499: mol_type = other DNA
gaacactgcgtttgctggctttggaagaaacatgagatcactgttctctctacagttactgagcacacaggacctcgccatgggatggagctgtatcattgtcctcttggtatcaacagctacaggtgtccactcccaggtccaactgcagcagcctggggctgagctggtgaggcctgggacttcagtgaagttgtcctgcaaggcttctggctacaccttcaccagctactggatacactgggtaaagcagaggcctggacaaggccttgagtggatcggactgattgatccttctgatagttatactaactacaatcaaaagttcaggggcaaggccacattgactgtagacacatcctccagcacagcctacatgcagctcagcagcctgacatctgaggactctgcggtctattactgtgcaagctccgatcctacaggctgctggggccaaggcaccactctcacagtctccccagctagcacaacaccccca

SEQ ID NO: 54 - DNA, 48 bp, Homo sapiens
  source 1..48: mol_type = other DNA
aggtctagtaagagtctcctacatagtagtggcatcacttatttgtct

SEQ ID NO: 55 - DNA, 20 bp, Homo sapiens
  source 1..20: mol_type = other DNA
cagatgtccaaccttgcctc

SEQ ID NO: 56 - DNA, 27 bp, Homo sapiens
  source 1..27: mol_type = other DNA
gctcaaaatctagaacttccgctcacg

SEQ ID NO: 57 - DNA, 30 bp, Homo sapiens
  source 1..30: mol_type = other DNA
ggctacaccttcaccagctactggatacac

SEQ ID NO: 58 - DNA, 51 bp, Homo sapiens
  source 1..51: mol_type = other DNA
ctgattgatccttctgatagttatactaactacaatcaaaagttcaggggc

SEQ ID NO: 59 - DNA, 18 bp, Homo sapiens
  source 1..18: mol_type = other DNA
tccgatcctacaggctgc

SEQ ID NO: 60 - DNA, 378 bp, Homo sapiens
  source 1..378: mol_type = other DNA
gaagtgcagctggtggagtctgggggaggcgcggtccaacctgggaggtccctgagactctcctgtgcagcctctggagtcagtttcagtaacattggcatgcactgggtccgccaggctccaggcaaggggctggagtgggtggcacttatatcatctgatggacgtcatacacactatgcagactccgtgaagggccgattcaccatctccagagacaattccgagaacacgctctatctacagatcaacggcctgagagctgacgacacggctgtttattactgtgcgaaaggccttgacactcgtgcccgttacatggggaaattttatacttttgactactggggccagggaaccctggtcaccgtctcctca

SEQ ID NO: 61 - DNA, 321 bp, Homo sapiens
  source 1..321: mol_type = other DNA
gacatccagttgacccagtctccatcctccctgtctgcatctgtaggagacagagtcaccatcacttgtcgggcaagtcagaacattggcgtcaatttaaattggtatcaacagagaccagggaaaggcccgaaactcctagtctcttctacatttagtttgcaaagtggggtcccatcaaggtttagtgccagtggagctgggacaaatttcactctcaccatcagcagtctgcaacctgaggactatgtcacttactactgtcaacagagttacagtagtccattcacattcggccctgggaccaaagtggatatcaaa

SEQ ID NO: 62 - protein, 14 aa, Homo sapiens
CGWTVLQKRIDGSL

SEQ ID NO: 63 - protein, 17 aa, Homo sapiens
CKKTTMKIIPFNRLTIG